(19) United States
(12) Patent Application Publication
(10) Pub. No.: US 2026/0096559 A1
(43) Pub. Date: Apr. 9, 2026

(54) MOLECULAR IODINE-INFUSED POLYMERS, ARTICLES. AND PRODUCTS, AND THEIR PREPARATION AND USE

(71) Applicant: I2PURE CORP., Ashburn, MA (US)

(72) Inventor: Jack KESSLER, Southborough, MA (US)

(73) Assignee: I2PURE CORP., Ashburn, MA (US)

(21) Appl. No.: 19/417,770

(22) Filed: Dec. 12, 2025

Related U.S. Application Data
(63) Continuation of application No. PCT/US2024/034220, filed on Jun. 14, 2024.
(60) Provisional application No. 63/508,268, filed on Jun. 14, 2023.

(51) Int. Cl. A01N 59/12; A01N 25/10; A01P 1/00; A61K 33/18; A61K 47/34; A61L 2/23; A61L 101/46; A61L 103/06; A61L 103/15; C08G 77/385

(52) U.S. Cl. CPC A01N 59/12; A01N 25/10; A01P 1/00; A61K 33/18; A61K 47/34; A61L 2/23; C08G 77/385; A61L 2101/46; A61L 2103/06; A61L 2103/15

(57) ABSTRACT

Provided herein are embodiments of molecular iodine-infused polymers, articles, and products, preparation of same, and uses of same.

### CROSS-REFERENCE TO RELATED APPLICATIONS

This application is a continuation of International Patent Application No. PCT/US2024/034220, filed Jun. 14, 2024, which claims the benefit of U.S. Provisional Patent Application No. 63/508,268, filed Jun. 14, 2023, both of which are incorporated herein by reference in their entirety.

### TECHNICAL FIELD

This invention relates to molecular iodine-infused polymers, articles, and products, and their preparation and use.

### BACKGROUND

Polymers are widely used in medical devices, e.g., catheters. Polymer based or associated infections remain a significant issue in healthcare. For example, about 40% of infections in healthcare are urinary tract infection (UTI), and 80% of UTI is triggered by catheter placement. More than 10% of the adult patients used indwelling catheters during a stay in the hospital, and 3% to 7% of patients had catheter-associated UTI. See, e.g., www.ncbi.nlm.nih.gov/pmc/articles/PMC6953942/#refl.

In this application, we describe molecular iodine-infused polymers, articles and products, and their preparation and use, e.g., in prevention and treatment of infections.

### SUMMARY

### Overview

The present disclosure is directed to molecular iodine-infused polymers, molecular iodine-infused articles and molecular iodine-infused products, and their preparation and use.

Embodiments of the molecular iodine-infused polymers are disclosed. The molecular iodine-infused polymer comprises a polymer and molecular iodine, and releases molecular iodine. Molecular iodine is released from the molecular iodine-infused polymer. The molecular iodine-infused polymer is prepared by mixing a high molecular iodine concentration composition with a pre-polymerization composition of the polymer to provide an iodine-pre-polymerization mixture, and then polymerizing the iodine-pre-polymerization mixture to provide the molecular iodine-infused polymer. The pre-polymerization composition may be a dispersion (e.g., a solution) comprising one or more monomers, one or more oligomers, and any combinations thereof.

Embodiments of the molecular iodine-infused articles are disclosed. In certain embodiments, the molecular iodine-infused article comprises one or more molecular iodine-infused polymers. In certain embodiments, the one or more molecular iodine-infused polymers form a coating of the article. In certain embodiments, the molecular iodine-infused article is prepared from one or more molecular iodine-infused polymers. In certain embodiments, a preparation method of the molecular iodine-infused article comprises preparing one or more portions of the molecular iodine-infused article with one or more iodine-pre-polymerization mixtures (e.g., through casting, extruding, etc.), and polymerizing the one or more iodine-pre-polymerization mixtures. Examples of the articles of molecular iodine-infused articles include, without limitation, catheters, sutures, grafts, stents, wound dressing material, bandage, artificial skin, implants, packaging to hold sterile medical materials, and polymers that provide a disinfecting atmosphere for materials (e.g., metals, polymers, fabrics, plants, and food) by virtue of being packaged with or placed adjacent to said materials.

Embodiments of the molecular iodine-infused products are disclosed. In certain embodiments, the molecular iodine-infused product comprises one or more molecular iodine-infused articles in one or more compartments.

In certain embodiments, the molecular iodine-infused product is a sanitizing chamber comprising a first compartment made of one or more molecular iodine-infused polymers. Molecular iodine released from the molecular iodine-infused polymers provides a disinfecting atmosphere for materials (e.g., metals, polymers, fabrics, plants, and food) may disinfect an article that is placed in the sanitizing chamber. In certain embodiments, the first compartment further includes one or more molecular iodine-infused polymers or articles therein. In certain embodiments, the first compartment includes one or more molecular iodine-infused polymers or articles therein but the first compartment itself is not made of a molecular iodine-infused polymer. In certain embodiments, the sanitizing chamber further comprises a second compartment separated from the first compartment by and a physical barrier. The barrier can be breakable to allow fluid communication between the first and second compartments.

### Biostatic Persistent, Biocidal Persistent, Prolonged Biocidal Activity, and Iodine Flux Rate

Molecular iodine is released from the molecular iodine-infused polymer or article. In certain embodiments, the molecular iodine-infused polymer or article is biostatic persistent. In certain embodiments, the molecular iodine-infused polymer or article is biocidal persistent. In certain embodiments, the molecular iodine-infused polymer or article has prolonged biocidal activity.

### Molecular Iodine Concentrations of the Molecular Iodine-Infused Polymer or Article and Stability

In certain embodiments, the molecular iodine-infused polymer or article has a molecular iodine mass concentration characterized by the mass of molecular iodine per unit mass of the molecular iodine-infused polymer or article.

In certain embodiments, the molecular iodine-infused polymer or article has a molecular iodine volume concentration characterized by the mass of molecular iodine per unit volume of the molecular iodine-infused polymer or article.

In certain embodiments, the molecular iodine flux rate of the molecular iodine-infused polymer or article drops no more than about 90%, about 85%, about 80%, about 75%, about 70%, about 65%, about 60%, about 55%, about 50%, about 10%, and about 1.0%, after exposure to ambient environment for a time period.

In certain embodiments, the molecular iodine mass concentration of the molecular iodine-infused polymer or article drops no more than about 90%, about 85%, about 80%, about 75%, about 70%, about 65%, about 60%, about 55%, about 50%, about 10%, and about 1.0%, after exposure to ambient environment for a time period.

In certain embodiments, the time period is about 5 minutes (min), about 10 min, about 15 min, about 20 min, about 25 min, about 30 min, about 35 min, about 40 min, about 45 min, about 50 min, about 55 min, about 1 hour (hr), about 1.5 hr, about 2 hr, about 2.5 hr, about 3 hr, about 3.5 hr, about 4 hr, about 4.5 hr, about 5 hr, about 5.5 hr, about 6 hr, about 6.5 hr, about 7 hr, about 7.5 hr, about 8 hr, about 8.5 hr, about 9 hr, about 9.5 hr, about 10 hr, about 10.5 hr, about 11 hr, about 11.5 hr, about 12 hr, about 12.5 hr, about 13 hr, about 13.5 hr, about 14 hr, about 14.5 hr, about 15 hr, about 15.5 hr, about 16 hr, about 16.5 hr, about 17 hr, about 17.5 hr, about 18 hr, about 18.5 hr, about 19 hr, about 19.5 hr, about 20 hr, about 20.5 hr, about 21 hr, about 21.5 hr, about 22 hr, about 22.5 hr, about 23 hr, about 23.5 hr, about 1 day (d), about 1.5 d, about 2 d, about 2.5 d, about 3 d, about 3.5 d, about 4 d, about 4.5 d, about 5 d, about 5.5 d, about 6 d, about 6.5 d, about 7 d, about 7.5 d, about 8 d, about 8.5 d, about 9 d, about 9.5 d, about 10 d, about 10.5 d, about 11 d, about 11.5 d, about 12 d, about 12.5 d, about 13 d, about 13.5 d, about 14 d, about 14.5 d, about 15 d, about 15.5 d, about 16 d, about 16.5 d, about 17 d, about 17.5 d, about 18 d, about 18.5 d, about 19 d, about 19.5 d, about 20 d, about 20.5 d, about 21 d, about 21.5 d, about 22 d, about 22.5 d, about 23 d, about 23.5 d, about 24 d, about 24.5 d, about 25 d, about 25.5 d, about 26 d, about 26.5 d, about 27 d, about 27.5 d, about 28 d, about 28.5 d, about 29 d, about 29.5 d, about 30 d, about 30.5 d, about 31 d, about 1.5 months, about 2 months, about 2.5 months, about 3 months, about 3.5 months, about 4 month, about 4.5 months, about 5 months, about 5.5 months, about 6 months.

### The High Molecular Iodine Concentration Compositions

In certain embodiments, the high molecular iodine concentration composition comprises molecular iodine at a concentration of about 100 ppm to about 1,120 ppm, about 300 ppm to about 1,274 ppm, about 1,274 ppm to about 170,000 ppm, about 1,274 ppm to about 160,000 ppm, about 1,274 ppm to about 150,000 ppm, about 1,274 ppm to about 100,000 ppm, about 1,274 ppm to about 66,000 ppm, about 1,274 ppm to about 66,000 ppm, about 1,274 ppm to about 15,200 ppm, about 1,400 ppm to about 68,000 ppm, about 1,400 ppm to about 15,200 ppm, about 1,400 ppm to about 66,000 ppm, about 1,500 ppm by weight/volume (wt/v), about 14,000 ppm to about 40,000 ppm, about 15,000 ppm to about 40,000 ppm, about 16,000 ppm to about 38,000 ppm, or about 14,000 ppm to about 20,000 ppm; and an organic carrier having a concentration of no less than about 93.5% wt, no less than about 95% wt, no less than about 98% wt, or no less than about 99% wt of the total weight of the high molecular iodine concentration composition.

In certain embodiments, the organic carrier of the high molecular iodine concentration composition is a pharmaceutically acceptable organic carrier, and the high molecular iodine concentration composition is a pharmaceutical formulation. In certain embodiments, the high molecular iodine concentration pharmaceutical formulation further comprises a second pharmaceutically acceptable carrier and may contain other ingredients to improve stability or impart other characteristics to the product (e.g. lubricity). In certain embodiments, the organic carrier has a boiling point at least about 250° C. In certain embodiments, the organic carrier is a weak solvent for the polymer and/or the pre-polymerization composition. In certain embodiments, the organic carrier swells the polymer and/or the pre-polymerization composition when in contact.

### Uses

Embodiments of a method of treating or preventing a condition associated with a microorganism in a subject comprising contacting the subject with one or more molecular iodine-infused polymers or articles disclosed herein. In certain embodiments, the microorganism is killed at the contact site or in vicinity of the contact site. In certain embodiments, the growth rate of the microorganism is reduced at the contact site or in vicinity of the contact site. In certain embodiments, the method further comprises delivering a therapeutically effective amount or prophylactically effective amount of molecular iodine, high molecular iodine concentration composition, or high molecular iodine concentration pharmaceutical formulation via the molecular iodine infused polymers or articles. In certain embodiments, the microorganism is present at the contact site or in vicinity of the contact site in or on the subject. In certain embodiments, a biofilm is present at the contact site or vicinity of the contact site. In certain embodiments, more than one species of microorganism are present at the contact site or vicinity of the contact site.

Examples of the conditions treatable or preventable include, without limitation, infections caused by microorganism, e.g., urinary tract infection, breast implant infections, and wound infection. In certain embodiments, the infection is chronic infection. In certain embodiments, the infection contains microorganisms that are resistant to antibiotics. In certain embodiments, the infection is a chronic urinary tract infection.

Provided herein are embodiments of method of fostering wound-healing or preventing scar of a wound of a subject comprising contacting the wound with one or more of the molecular iodine-infused polymers or molecular iodine-infused articles (e.g., wound dressing and artificial skin) or molecular iodine-infused products.

Provided herein are embodiments of a method of treating a surgical site to foster healing, or prevent infection in a subject in need comprising contacting the subject with the molecular iodine-infused polymer or molecular iodine-infused article (e.g., wound dressing and artificial skin), e.g., at the surgical site. In certain embodiments, the method of treating a surgical site is performed as a pre-surgical treatment, a post-surgical treatment, or a treatment during a surgery.

Provided herein are embodiments of sanitizing one or more articles or materials (e.g., metals, polymers, fabrics, plants, and food) comprising placing the one or more articles or materials adjacent to one or more molecular iodine-infused polymers or one or more molecular iodine-infused articles. In certain embodiments, the one or more articles or materials are placed in a sealed compartment comprising the one or more molecular iodine-infused polymers or one or more molecular iodine-infused articles. In certain embodiments, the sealed compartment is made of the one or more molecular iodine-infused polymers. In certain embodiments, the sealed compartment comprises one or more molecular iodine-infused polymers or articles placed therein.

### BRIEF DESCRIPTION OF THE DRAWINGS

This application contains at least one drawing executed in color. Copies of this application with color drawing(s) will be provided by the Office upon request and payment of the necessary fees.

FIG. 1: An embodiment of the molecular iodine-infused silicone prepared by Example 2 (right) and a silicone that does not comprise molecular iodine (left).

FIG. 2: An embodiment of the molecular iodine-infused silicone prepared by Example 2 after 24 hours exposure under ambient condition (left) and a silicone that does not comprise molecular iodine (left).

FIG. 3: Images of embodiments of the molecular iodine-infused silicone, samples 3(A)-A (1 wt % I₂) and 3(A)-B (0.1 wt % I₂) prepared using I₂/glycerin solution (10 wt % I₂) according to Example 3(A) as prepared and after placed in a sealed environment (top panel) or open to air (bottom panel) for Day 1, 2, 3, 5, or 7.

FIG. 4: Embodiments of the molecular iodine-infused silicone, samples 3(B)-A (10 wt % I₂) and 3(B)-B (1 wt % I₂) prepared using powdered I₂ according to Example 3(B). (A): Images of samples 3(B)-A and 3(B)-B in sealed dishes as prepared or after day 1. (B): Images of samples 3(B)-A and 3(B)-B when exposed to air for 10 min, 2 h, 4 h, or 24 h. (C): Kinetics of I2 loss from samples 3(B)-A (the left bar in blue) and 3(B)-B (the right bar in orange) by measurement of the samples' I₂ concentrations over time. The net change is also affected by loss of alcohols during cure, which were not measured directly. However, I₂ can easily dissolve in alcohols and will be carried away by evaporation. Unless specified otherwise, wt % I₂ in a molecular iodine-infused silicone is measured by the mass of I₂ over the total mass of the molecular iodine-infused silicone.

FIG. 5: An embodiment of the molecular iodine-infused silicone, sample 3(C) (50 wt % I₂) prepared using powdered I₂ according to Example 3(C). (A): Images of sample 3(C) in sealed dishes (top panel) or open to air (bottom panel) as prepared or after day 1, 2, or 8. (B): Images of sample 3(C) showing preferential migration of I₂ from the elastomer edges when exposed to air after 8 days. (C): Kinetics of I2 loss from sample 3(C) after exposed to air after 8 days. Samples 3(C) were 21.45 mm in diameter and 3.12 mm thick and placed into 34.6 mm Petri dishes.

FIG. 6. Embodiments of the molecular iodine-infused silicone, samples 3(D)-A (low crosslink density silicone) and 3(D)-B (high crosslink density silicone) prepared using a single I₂ crystals placed between two layers of precured silicones according to Example 3(D). (A): A schematic representation of Sample 3(D). (B): Sample 3(D)-A placed in a Ziploc bag for 5 min. (C): Sample 3(D)-A placed in a Ziploc bag for 48 h. (D): Sample 3(D)-B placed in a Ziploc bag for 5 min. (E): Sample 3(D)-B placed in a Ziploc bag for 48 h. (F): Sample 3(D)-A placed in a sealed Petri dish for 5 min. (G): Sample 3(D)-A placed in a sealed Petri dish for 48 h. (H): Sample 3(D)-B placed in a sealed Petri dish for 5 min. (I): Sample 3(D)-B placed in a sealed Petri dish for 48 h. (J): Sample 3(D)-A placed in an open Petri dish for 5 min. (K): Sample 3(D)-A placed in an open Petri dish for 48 h. (L): Sample 3(D)-B placed in an open Petri dish for 5 min. (M): Sample 3(D)-B placed in an open Petri dish for 48 h. Sample edges shown with blue lines.

FIG. 7. Additional embodiments of the molecular iodine-infused silicone, samples 3(D)-A (low crosslink density silicone) and 3(D)-B (high crosslink density silicone) prepared using a single I₂ crystals placed between two layers of precured silicones according to Example 3(D). (A): Sample 3(D)-A (with 9.7% mass increase in the two silicone pieces, and at equilibrium, the iodine mass in the two silicone pieces accounting for 8.8% of the initial solid iodine placed between the two silicone pieces) placed in a Ziploc bag for 5 min. 30 min, 1 h, 4 h, 8 h, 24 h, 48 h, 72 h, and 7 days. (B): Sample 3(D)-B (with 10.0% mass increase in the silicone, and at equilibrium, the iodine mass in the two silicone pieces accounting for 11.2% of the initial solid placed between the two silicone pieces) placed in a Ziploc bag for 5 min. 30 min, 1 h, 4 h, 8 h, 24 h, 48 h, 72 h, and 7 days.

FIG. 8. An embodiment of the molecular iodine-infused silicone, sample 3(E)-A (low crosslink density silicone) prepared using multiple I₂ crystals placed between two layers of precured silicones according to Example 3(E). (A): Sample 3(E)-A placed in a Ziploc bag for 4 h. (B): Sample 3(E)-A placed in a sealed Petri dish for 4 h. (C): Sample 3(E)-A placed in a sealed Petri dish for 4 h.

FIG. 9: Kinetics of I₂ loss from high crosslink density silicone treated with single crystal or multiple I₂ crystals. (A) I2 concentration in high crosslink density silicone treated with single I₂ crystal at time (t)=0 and 1 week and (B) I2 concentration in high crosslink density silicone treated with multiple I₂ crystal at t=0 and 1 week.

FIG. 10: Embodiments of the molecular iodine-infused silicone, samples prepared by higher I₂ ratio treatment (Rubber:I₂=1:1) and lower I₂ ratio treatment (Rubber:I₂=10:1) were prepared using I₂ vapor according to Example 3(F) for high and low crosslink silicones, respectively. (A): Exemplary images of samples with lower I₂ ratio treatment (Rubber:I₂=10:1) and higher I₂ ratio treatment (Rubber:I₂=1:1) placed in a sealed Petri dish after 30 min, day 1, 2, 3, or 6. (B): I₂ increases in low crosslink density silicone with higher I₂ ratio treatment (1:1 Low, dark circle), low crosslink density silicone with lower I₂ ratio treatment (10:1 Low, diamond), high crosslink density silicone with higher I₂ ratio treatment (1:1 High, triangle), and high crosslink density silicone with lower I₂ ratio treatment (10:1 high, light circle) after days of exposure to I₂ vapor.

FIG. 11: Embodiments of the molecular iodine-infused silicone sample prepared using I₂ vapor according to Example 3(G). (A): Thin film placed in a Ziploc bag after 5 min with the presence of an I₂ crystal not in direct contact with the sample. (B): Thin film placed in a Ziploc bag after 48 h with the presence of an I₂ crystal not in direct contact with the sample. (C): Thin film placed in a sealed petri dish after 5 min with the presence of an I₂ crystal not in direct contact with the sample. (D): Thin film placed in a sealed petri dish after 48 h with the presence of an I₂ crystal not in direct contact with the sample. Edges of the sample are shown with blue lines.

FIG. 12: Loss of I₂ from the samples of FIG. 10(A) over a few hours. (A): Images of samples with higher I₂ ratio treatment (Rubber:I₂=1:1) and lower I₂ ratio treatment (Rubber:I₂=10:1) after opened to air for 2 h, 4 h, or 24 h. (B): Bar chart showing the loss of I₂ from samples with higher I₂ ratio treatment (Rubber:I₂=1:1, left bar) and lower I₂ ratio treatment (Rubber:I₂=10:1, right bar) after opened to air for 2 h, 4 h, or 24 h.

### DETAILED DESCRIPTION

The molecular iodine-infused polymers, articles, and products disclosed herein are especially useful for treating or preventing infection when the articles or polymers need to be kept in a subject for days, weeks, months, or longer. For example, about 40% of infections in healthcare are urinary tract infection (UTI), and 80% of UTI is triggered by catheter placement. More than 10% of the adult patients used indwelling catheters during a stay in the hospital, and 3% to 7% of patients had catheter-associated UTI. See, e.g., www.ncbi.nlm.nih.gov/pmc/articles/PMC6953942/#refl. The Examples section provides embodiments of the molecular iodine-infused polymers prepared by polymerization of an embodiment of an iodine-pre-polymerization mixture and partial loss of molecular iodine after exposure to ambient environment for 24 hours (Example 2).

### Definitions

The singular forms “a,” “an,” and “the” include plural referents unless the context clearly dictates otherwise. Thus, for example, reference to an “excipient” includes a single excipient as well as two or more different excipients, and the like.

The term “comprising,” which is inclusive or open-ended and does not exclude additional unrecited elements or method steps, is intended to encompass as alternative embodiments, the phrases “consisting essentially of” and “consisting of” where “consisting of” excludes any element or step not specified and “consisting essentially of” permits the inclusion of additional unrecited elements or steps that do not materially affect the essential or basic and novel characteristics of the composition or method under consideration.

The term “optional” or “optionally” means that the subsequently described circumstance may or may not occur, so that the description includes instances where the circumstance occurs and instances where it does not.

The term “pharmaceutically acceptable” in reference to an entity or ingredient is one that causes no significant adverse toxicological effects in a subject when administered to the subject.

The term “silicone” refers to a silicone or silicone rubber. In certain embodiments, the silicone is medical grade silicone. In certain embodiments, silicone comprises a polysiloxane having a chemical formula [R₁(R₂)SiO]ₙ, where R₁ and R₂ are independently selected from the group consisting of substituted and unsubstituted alkyl (e.g., C₁-C₂₀, e.g., methyl, ethyl), substituted and unsubstituted aryl (e.g., phenyl), substituted and unsubstituted alkylaryl, and substituted and unsubstituted arylalkyl. The silicone (e.g., silicone rubber) can be free of or substantially free of other resins or polymers. Pure silicone (e.g., silicone rubber) refers to commercially available silicones (e.g., silicone rubbers) that do not include other monomers or polymers or that include only trace or incidental amounts of other monomers or polymers.

The term “molecular iodine” refers to diatomic iodine, which is represented by the chemical symbol I₂ (CAS Registry Number: 7553-56-2) whether dissolved, suspended or in a solid state. The term “molecular iodine” may also be referred to as “elemental iodine” when in the solid state and is sometimes represented as “I₂” in this application. The term “molecular iodine” may also be referred as “I₂,” “free molecular iodine,” “unbound molecular iodine,” “uncomplexed molecular iodine,” and “un-complexed molecular iodine” in the art. Chemical activity of molecular iodine is not reduced by association with or complexation with other polymers or iodide and therefore exhibits antimicrobial activity. In aqueous solutions only hypoiodious acid (HOI) and molecular iodine are biocidal. The active biocide in acidic iodine-based biocides is believed to be free molecular iodine.

The term “iodine flux” or “iodine flux rate” refers to the release of molecular iodine from a polymer. Quantitative measurements of the iodine flux are provided in units of the mass (e.g., μg I₂) of molecular iodine released from a polymer per unit time (e.g., seconds) per unit area of the polymer (e.g., cm²) or unit mass (e.g., grams).

The term “iodide” or “iodide anion” refers to the species which is represented by the chemical symbol I⁻ (CAS Registry Number: 20461-54-5). Suitable counter-ions for the iodide anion include sodium, potassium, calcium, and the like.

The term “all iodine species” in a sample refers to all iodine containing components in the sample.

The term “ratio of molecular iodine to all iodine species” in a composition refers to the iodine content of molecular iodine (I₂) in the composition divided by the iodine content of all iodine species in the composition.

The term “organic carrier” refers to an organic molecule in which molecular iodine can be dispersed and the organic molecule does not react with molecular iodine. Examples of organic carrier include glycols with molecular weight below 300 (e.g., propylene glycol, di-propylene glycol, glycerin), propylene glycol monomethyl ether acetate, dimethyl sulfoxide (DMSO), alcohols (e.g., ethanol, propanols such as isopropanol and 1-propanol), and any mixtures of the foregoing.

The term “stable” means that the variation of the molecular iodine content of the molecular iodine-infused polymers, articles, or products, or the high molecular iodine concentration composition or pharmaceutical formulation is less than 10% of the initial molecular iodine content.

The term “shelf-life” means the time period that the molecular iodine-infused polymers, articles, or products, the high molecular iodine concentration composition or pharmaceutical formulation remains stable in a package under a storage condition. In certain embodiments, the shelf-life is at least 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, or 5 years.

The term “effective amount” means an amount of the high molecular iodine concentration composition or pharmaceutical formulation that is needed to effectuate a desired clinical outcome from a subject the high molecular iodine concentration composition or pharmaceutical formulation is administered to.

The term “biostatic persistence” means that after the subject's contact with the molecular iodine-infused polymer or article or the high molecular iodine concentration composition or pharmaceutical formulation, the microorganism count at the contact site will be equal to or lower than baseline for a first time period post contact. The term “baseline” means the microorganism count at the site of contact immediately before contact of the molecular iodine-infused polymer or article or the high molecular iodine concentration composition or pharmaceutical formulation.

Unless otherwise specified, the term “biocidal persistence” means that at the subject's contact site of the molecular iodine-infused polymer or article or the high molecular iodine concentration composition or pharmaceutical formulation, wherein the site is subjected to a first microorganism challenge, the microorganism count reduction after a second time period post contact is at least about 1 log lower than the microorganism count at a site of the same microorganism challenge without the contact. The term “biocidal persistence” may be modified by the extent of microorganism reduction and the second time period the “biocidal persistence” lasts. For example, a 3 log/6 hours biocidal persistence means that at the site of the contact of the molecular iodine-infused polymer or article or the high molecular iodine concentration composition or pharmaceutical formulation the microorganism count measured at six hours post contact is reduced by about 3 log compared to the microorganism count at a site of the same microorganism challenge without the contact six hours post challenge. For example, a 2 log/12 hour biocidal persistence means that at the site of the contact of the molecular iodine-infused polymer or article or the high molecular iodine concentration composition or pharmaceutical formulation the microorganism count measured twelve hours post contact is reduced by about 2 log lower compared to the microorganism count at a site of the same microorganism challenge without the contact 12 hours later.

Unless otherwise specified, the term “prolonged biocidal activity” means that at the site of the contact of the molecular iodine-infused polymer or article or the high molecular iodine concentration composition or pharmaceutical formulation, wherein the site is subject to the first microorganism challenge and at least a second microorganism challenge during a third time period post contact, the microorganism count measured at the end of the third time period post contact is reduced at least about 1 log compared to the microorganism count at a site subjected to the same microorganism challenges at the end of the third time period. Similarly, the term prolonged biocidal activity may be further defined by the extent of microorganism reduction and the third time period the “prolonged biocidal activity” lasts. For example, a 3 log/6 hours prolonged biocidal activity means that at the site of the contact of molecular iodine-infused polymer or article or the high molecular iodine concentration composition or pharmaceutical formulation, the microorganism count measured six hours post contact is reduced about 3 log compared to the microorganism count at a site of the same microorganism challenges after 6 hours without the contact, wherein other than the first microorganism challenge, at least a second microorganism challenge is imposed within 6 hours post contact. For example, a 2 log/12 hour prolonged biocidal activity means that at the site of the contact of molecular iodine-infused polymer or article or the high molecular iodine concentration composition or pharmaceutical formulation, the microorganism count measured 12 hours post contact is reduced about 2 log compared to the microorganism count at a site of the same microorganism challenges after 12 hours without the contact, wherein other than the first microorganism challenge, at least a second microorganism challenge is imposed within 12 hours post contact.

The term “non-aqueous” means the total water content of the high molecular iodine concentration composition or pharmaceutical formulation is no more than 1.5% wt of the total weight of the composition.

The term “substantially non-aqueous” means the total water content of the high molecular iodine concentration composition or pharmaceutical formulation is no more than 5.0% wt of the total weight of the composition.

The term “water-free” means the total water content of the high molecular iodine concentration composition or pharmaceutical formulation is no more than 0.5% wt of the total weight of the composition.

The term “substantially water-free” means the total water content of the high molecular iodine concentration composition or pharmaceutical formulation is no more than 2.5% wt of the total weight of the composition.

The term “alcohol-free” means the total alcohol content of the high molecular iodine concentration composition or pharmaceutical formulation is no more than 0.75% wt of the total weight of the composition.

The term “substantially alcohol-free” means the total alcohol content of the high molecular iodine concentration composition or pharmaceutical formulation is no more than 5.5% wt of the total weight of the composition.

The term “PPM” of “ppm” of an agent in a composition means a weight/volume (wt/v) concentration of the agent in the composition expressed in parts per million, which may also be referred to as a mass/volume (m/v) concentration.

The term “surgical site” means an incision site on a subject, or any part of a subject's anatomy in organs or spaces which is opened or manipulated during a surgery.

The term “about” before a numeric value means a range of 10% of the numeric value.

The present disclosure is directed to molecular iodine-infused polymers, molecular iodine-infused articles, and molecular iodine-infused products, and their preparation and use.

### I. Molecular Iodine-Infused Polymers

Embodiments of the molecular iodine-infused polymer are disclosed herein. The molecular iodine-infused polymer comprises a polymer and molecular iodine, and releases molecular iodine. In certain embodiments, the molecular iodine-infused polymer is prepared by mixing a high molecular iodine concentration composition with a pre-polymerization composition of the polymer to provide an iodine-pre-polymerization mixture, and then polymerizing the iodine-pre-polymerization mixture to provide the molecular iodine-infused polymer. The pre-polymerization composition may be a dispersion (e.g., a solution) comprising one or more monomers, one or more oligomers, and any combinations thereof. In certain embodiments, the molecular iodine-infused polymer is prepared by iodinating the polymer. In certain embodiments, iodinating the polymer comprises one or more steps selected from the following: mixing a high molecular iodine concentration composition with the polymer, contacting one or more pieces of solid iodine with the polymer, and contacting an iodine vapor with the polymer. In certain embodiments, the molecular iodine-infused polymer is prepared by iodinating one or more pre-polymerization compositions of the polymer or article to provide one or more iodine-pre-polymerization mixtures, and then polymerizing the one or more iodine-pre-polymerization mixtures. In certain embodiments, iodinating the one or more pre-polymerization compositions of the polymer comprises one or more steps selected from the following: mixing a high molecular iodine concentration composition with one or more pre-polymerization compositions of the polymer, contacting one or more pieces of solid iodine with the one or more pre-polymerization compositions of the polymer, and contacting an iodine vapor with the one or more pre-polymerization compositions of the polymer.

In certain embodiments, the molecular iodine-infused polymer is prepared as described in section VI, “Preparation methods of embodiments of molecular iodine-infused polymer, article or product.”

In certain embodiments, the polymerization step comprises contacting the iodine-pre-polymerization mixture with one or more initiators that initiate and/or one or more catalysts that catalyze the polymerization. In certain embodiments, the polymer is silicone. In certain embodiments, the polymer is silicone and the polymerization step comprising contacting the iodine-pre-polymerization mixture with a platinum catalyst, e.g., as shown in Scheme 1A below. In certain embodiments, the polymer is a RTV silicone prepared as shown in Scheme 1B below. In certain embodiments, the molecular iodine-infused polymer degrades in less than an hour. In certain embodiments, the molecular iodine-infused polymer does not degrade at least after 1 day.

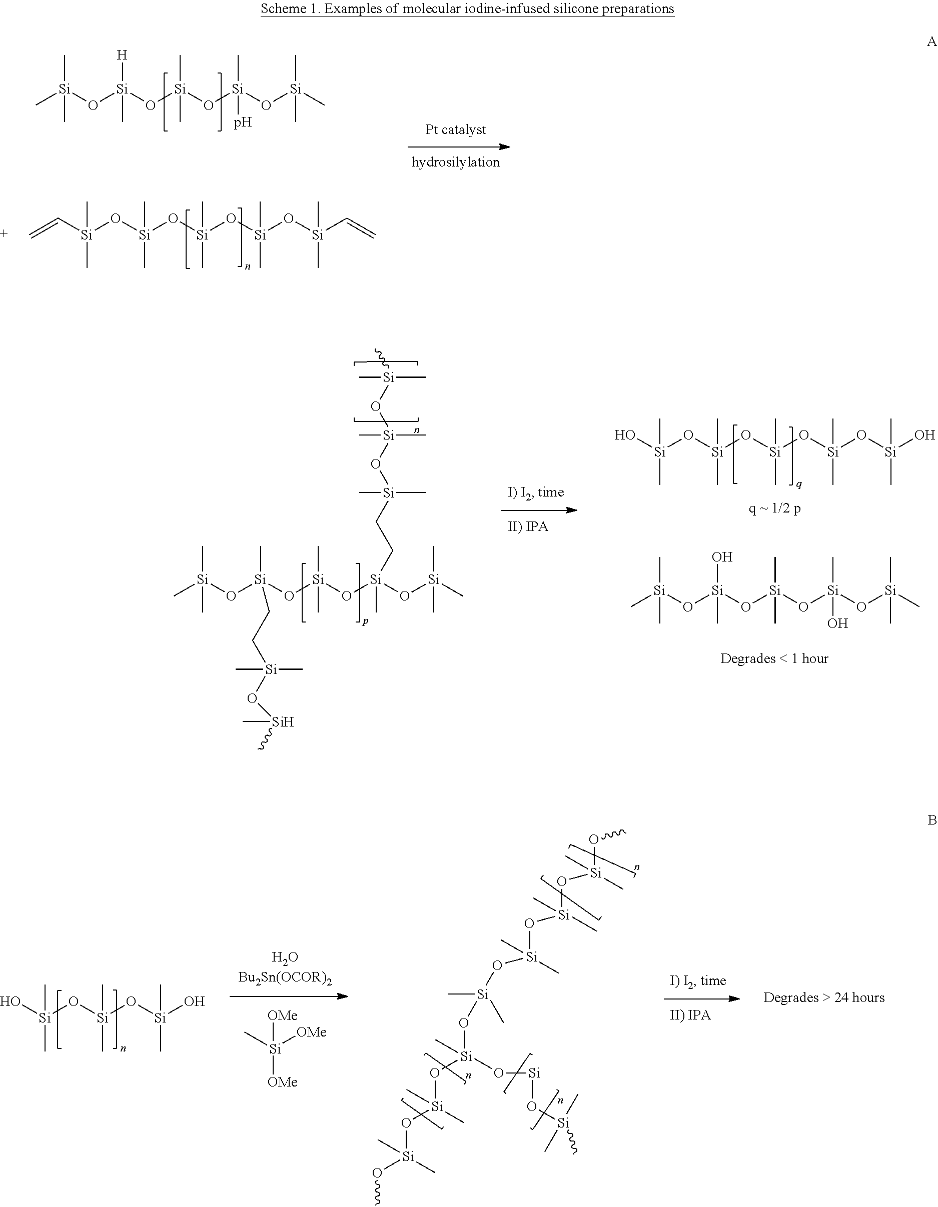

In certain embodiments, the silicone is room temperature vulcanizing silicones (RTV silicones). In certain embodiments, the RTV silicones are medical grade RTV silicones.

In certain embodiments, the silicone is a low crosslink density silicone. In certain embodiments, the low crosslink density silicone has a base to curing agent (e.g., catalyst) of about 10:0.4, about 10:0.5, or about 10:0.6. In certain embodiments, the low crosslink density silicone has a molecular weight between crosslinks of about 6 k to about 14 k g/mol. In certain embodiments, the low crosslink density silicone has an elastic modulus of about 0.5 to about 1.20 Mpa.

In certain embodiments, the silicone is a high crosslink density silicone. In certain embodiments, the high crosslink density silicone has a base to curing agent (e.g., catalyst) of about 10:0.8, or about 10:1. In certain embodiments, the high crosslink density silicone has a molecular weight between crosslinks of about 23 k to about 40 k g/mol. In certain embodiments, the high crosslink density silicone has an elastic modulus of about 1.8 to about 3.0 Mpa.

In certain embodiments, the molecular iodine-infused polymer comprises a first molecular iodine-infused region and a second molecular iodine-infused region. In certain embodiments, molecular iodine from the first molecular iodine-infused region diffuses to the second molecular iodine-infused region while molecular iodine of the second molecular iodine-infused region is released from the polymer.

In certain embodiments, the first molecular iodine-infused region has a higher molecular iodine concentration (either mass concentration or volume concentration) than the second molecular iodine-infused region and molecular iodine in the second molecular iodine-infused region is released faster than molecular iodine in the first molecular iodine-infused region. In certain embodiments, the first molecular iodine-infused region has a higher molecular iodine concentration than the second molecular iodine-infused region and molecular iodine in the first molecular iodine-infused region is released faster than molecular iodine in the second molecular iodine-infused region.

In certain embodiments, the polymer is a natural or synthetic polymer. Examples of natural polymers include, without limitation, natural latex, which may be further processed or treated. In certain embodiments, examples of the synthetic polymer include, without limitation, silicone, synthetic latex rubber, polyester (e.g., Dacron), and co-polymers thereof.

In certain embodiments, the polymers are medical-grade polymers. In certain embodiments, the molecular iodine-infused polymers are medical-grade polymers.

### II. Molecular Iodine-Infused Articles

Embodiments of the molecular iodine-infused articles are disclosed herein. In certain embodiments, the molecular iodine-infused article comprises one or more molecular iodine-infused polymers. In certain embodiments, the one or more molecular iodine-infused polymers form a coating of the article. In certain embodiments, the molecular iodine-infused article is prepared from one or more molecular iodine-infused polymers. In certain embodiments, a preparation method of the molecular iodine-infused article comprises preparing one or more portions of the molecular iodine-infused article with one or more iodine-pre-polymerization mixtures (e.g., through casting, extruding, etc.), and polymerizing the one or more iodine-pre-polymerization mixtures. In certain embodiments, the molecular iodine-infused article is prepared by iodinating the article. In certain embodiments, iodinating the article comprises one or more steps selected from the following: mixing a high molecular iodine concentration composition with the article, contacting one or more pieces of solid iodine with the article, and contacting an iodine vapor with the article. In certain embodiments, the molecular iodine-infused article is prepared by iodinating one or more pre-polymerization compositions of the article to provide one or more iodine-pre-polymerization mixtures, and then polymerizing the one or more iodine-pre-polymerization mixtures. In certain embodiments, iodinating the one or more pre-polymerization compositions of the article comprises one or more steps selected from the following: mixing a high molecular iodine concentration composition with one or more pre-polymerization compositions of the article, contacting one or more pieces of solid iodine with the one or more pre-polymerization compositions of the article, and contacting an iodine vapor with the one or more pre-polymerization compositions of the article. In certain embodiments, the molecular iodine-infused article is prepared as described in section VI, “Preparation methods of embodiments of molecular iodine-infused polymer, article or product.”

In certain embodiments, the articles are medical-grade articles. In certain embodiments, the molecular iodine-infused articles are medical-grade articles.

In certain embodiments, the molecular iodine-infused article comprises one or more molecular iodine-infused polymers that are medical-grade. In certain embodiments, the molecular iodine-infused article is for medical use. In certain embodiments, the molecular iodine-infused article is a medical device. Examples of the articles of molecular iodine-infused articles include, without limitation, catheters, sutures, grafts, stents, wound dressing material, bandage, artificial skin, implants, packaging to hold sterile medical materials, and polymers that provide a disinfecting atmosphere for materials (e.g., metals, polymers, fabrics, plants, and food) by virtue of being packaged with or placed adjacent to said materials.

Examples of molecular iodine-infused catheters include, without limitation, indwelling catheters, port catheters for, e.g., dialysis.

### III. Molecular Iodine-Infused Products

Embodiments of the molecular iodine-infused products are disclosed. In certain embodiments, the molecular iodine-infused product comprises one or more molecular iodine-infused articles in one or more compartments.

In certain embodiments, the molecular iodine-infused product is a sanitizing chamber comprising a first compartment made of one or more molecular iodine-infused polymers. Molecular iodine released from the molecular iodine-infused polymers provides a disinfecting atmosphere for materials (e.g., metals, polymers, fabrics, plants, and food) may disinfect an article that is placed in the sanitizing chamber. In certain embodiments, the first compartment further includes one or more molecular iodine-infused polymers or articles therein. In certain embodiments, the first compartment includes one or more molecular iodine-infused polymers or articles therein but the first compartment itself is not made of a molecular iodine-infused polymer. In certain embodiments, the sanitizing chamber further comprises a second compartment separated from the first compartment by and a physical barrier. The barrier can be breakable to allow fluid communication between the first and second compartments.

In certain embodiments, the compartments of the molecular iodine-infused product comprise one or more material that is dormant to molecular iodine infusion, e.g., without limitation, LLDPE, PTFE, and PET.

In certain embodiments, the molecular iodine-infused product is prepared as described in section VI, “Preparation methods of embodiments of molecular iodine-infused polymer, article or product.”

### IV. Biostatic Persistent, Biocidal Persistent, Prolonged Biocidal Activity, Iodine Flux Rate, Molecular Iodine Concentration of Molecular Iodine-Infused Polymers, and Articles, and Stability.

Molecular iodine is released from the molecular iodine-infused polymer or article.

In certain embodiments, the molecular iodine-infused polymer or article is biostatic persistent. In certain embodiments, the molecular iodine-infused polymer or article is biocidal persistent. In certain embodiments, the molecular iodine-infused polymer or article has prolonged biocidal activity. In certain embodiments, molecular iodine is released from the molecular iodine-infused polymer or article.

The molecular iodine-infused polymer or article has an iodine flux rate. In certain embodiments, the molecular iodine-infused polymer or article has an iodine flux rate sufficient to afford biostatic persistent, biocidal persistent, and prolonged biocidal activity.

In certain embodiments, the iodine flux rate of the molecular iodine-infused polymer or article drops no more than about 90%, about 85%, about 80%, about 75%, about 70%, about 65%, about 60%, about 55%, or about 50% after a time period.

Examples of the iodine flux rate include, without limitation, at least about 0.02, at least about 0.03, at least about 0.08, at least about 0.10, at least about 0.17, at least about 0.19, at least about 0.20, at least about 0.27, at least about 0.45, at least about 0.60, at least about 0.64, at least about 0.69, or at least about 1.08 μg molecular iodine per second per cm² of the molecular iodine-infused polymer or article. Examples of the iodine flux rate include, without limitation, at least about 0.0020, at least about 0.0039, at least about 0.0085, at least about 0.017, at least about 0.029, at least about 0.069, at least about 0.073, at least about 0.078, at least about 0.087, at least about 0.090, at least about 0.11, at least about 0.13, at least about 0.15, at least about 0.19, at least about 0.20, at least about 0.28, at least about 0.30, at least about 0.34, at least about 0.39, at least about 0.41, at least about 0.49, at least about 0.52, at least about 0.547, at least about 0.59, at least about 0.63, at least about 0.74, at least about 1.29, at least about 1.7, at least about 1.81, at least about 3.5, at least about 4.9, at least about 5.0, at least about 5.5, at least about 5.7, at least about 5.8, at least about 6.3, at least about 7.1, at least about 7.2, at least about 7.4, at least about 8.98, at least about 9.4, or at least about 10.94 μg molecular iodine per second per gram of the molecular iodine-infused polymer or article, or ranges therebetween. In certain embodiments, the molecular iodine-infused polymer or article has an iodine flux rate of 0.19 μg of I₂ per second per gram of polymer for at least about 5 hours, 24 hours, 10 days, or 10.4 days.

The molecular iodine-infused polymer or article has a molecular iodine mass concentration which may be characterized as the mass of molecular iodine per unit mass of the molecular iodine-infused polymer or article. Examples of the molecular iodine mass concentration includes, without limitation, at least about 13.0 mg/g, 5.16 mg/g, 4.54 mg/g. 2.32 mg/g, and ranges therebetween. In certain embodiments, the molecular iodine-infused polymer or article has a molecular iodine mass concentration shown in wt % of molecular iodine over the total weight of the molecular iodine-infused polymer or article. Examples of the molecular iodine mass concentration includes, without limitation, at least about 0.1 wt %, about 0.2 wt %, about 0.3 wt %, about 0.4 wt %, about 0.5 wt %, about 0.6 wt %, about 0.7 wt %, about 0.8 wt %, about 0.9 wt %, about 1.0 wt %, about 1.1 wt %, about 1.2 wt %, about 1.3 wt %, about 1.4 wt %, about 1.5 wt %, about 1.6 wt %, about 1.7 wt %, about 1.8 wt %, about 1.9 wt %, about 2.0 wt %, about 2.1 wt %, about 2.2 wt %, about 2.3 wt %, about 2.4 wt %, about 2.5 wt %, about 2.6 wt %, about 2.7 wt %, about 2.8 wt %, about 2.9 wt %, about 3.0 wt %, about 3.1 wt %, about 3.2 wt %, about 3.3 wt %, about 3.4 wt %, about 3.5 wt %, about 3.6 wt %, about 3.7 wt %, about 3.8 wt %, about 3.9 wt %, about 4.0 wt %, about 4.1 wt %, about 4.2 wt %, about 4.3 wt %, about 4.4 wt %, about 4.5 wt %, about 4.6 wt %, about 4.7 wt %, about 4.8 wt %, about 4.9 wt %, about 5.0 wt %, about 5.1 wt %, about 5.2 wt %, about 5.3 wt %, about 5.4 wt %, about 5.5 wt %, about 5.6 wt %, about 5.7 wt %, about 5.8 wt %, about 5.9 wt %, about 6.0 wt %, about 6.1 wt %, about 6.2 wt %, about 6.3 wt %, about 6.4 wt %, about 6.5 wt %, about 6.6 wt %, about 6.7 wt %, about 6.8 wt %, about 6.9 wt %, about 7.0 wt %, about 7.1 wt %, about 7.2 wt %, about 7.3 wt %, about 7.4 wt %, about 7.5 wt %, about 7.6 wt %, about 7.7 wt %, about 7.8 wt %, about 7.9 wt %, about 8.0 wt %, about 8.1 wt %, about 8.2 wt %, about 8.3 wt %, about 8.4 wt %, about 8.5 wt %, about 8.6 wt %, about 8.7 wt %, about 8.8 wt %, about 8.9 wt %, about 9.0 wt %, about 9.1 wt %, about 9.2 wt %, about 9.3 wt %, about 9.4 wt %, about 9.5 wt %, about 9.6 wt %, about 9.7 wt %, about 9.8 wt %, about 9.9 wt %, about 10.0 wt %, about 10.1 wt %, about 10.2 wt %, about 10.3 wt %, about 10.4 wt %, about 10.5 wt %, about 10.6 wt %, about 10.7 wt %, about 10.8 wt %, about 10.9 wt %, about 11.0 wt %, about 12.0 wt %, about 13.0 wt %, about 14.0 wt %, about 15.0 wt %, about 16.0 wt %, about 17.0 wt %, about 18.0 wt %, about 19.0 wt %, about 20.0 wt %, about 21.0 wt %, about 22.0 wt %, about 23.0 wt %, about 24.0 wt %, about 25.0 wt %, about 26.0 wt %, about 27.0 wt %, about 28.0 wt %, about 29.0 wt %, about 30.0 wt %, about 31.0 wt %, about 32.0 wt %, about 33.0 wt %, about 34.0 wt %, about 35.0 wt %, about 36.0 wt %, about 37.0 wt %, about 38.0 wt %, about 39.0 wt %, about 40.0 wt %, about 41.0 wt %, about 42.0 wt %, about 43.0 wt %, about 44.0 wt %, about 45.0 wt %, about 46.0 wt %, about 47.0 wt %, about 48.0 wt %, about 49.0 wt %, about 50.0 wt %, about 51.0 wt %, about 52.0 wt %, about 53.0 wt %, about 54.0 wt %, about 55.0 wt %, about 56.0 wt %, about 57.0 wt %, about 58.0 wt %, about 59.0 wt %, about 60.0 wt %, about 61.0 wt %, about 62.0 wt %, about 63.0 wt %, about 64.0 wt %, about 65.0 wt %, about 66.0 wt %, about 67.0 wt %, about 68.0 wt %, about 69.0 wt %, about 70.0 wt %, and ranges therebetween.

The molecular iodine-infused polymer or article has a molecular iodine volume concentration which may be characterized as the mass of molecular iodine per unit volume of the molecular iodine-infused polymer or article.

In certain embodiments, the molecular iodine-infused polymer or article has one or more biocidal effects selected from the group consisting of biostatic persistent, biocidal persistent, and prolonged biocidal activity for a time period. In certain embodiments, the molecular iodine-infused polymer or article has an iodine flux rate sufficient to afford one or more biocidal effects selected from the group consisting of biostatic persistent, biocidal persistent, and prolonged biocidal effects for a time period. In certain embodiments, the molecular iodine-infused polymer or article has an iodine flux rate or a molecular iodine concentration for a time period. Examples of the time period include, without limitation, about 5 hour to about 11 days, about 24 hours to about 2 weeks, about 10 days to about 2 weeks, at least about 1 min, 5 min, 15 min, 30 min, 1 hr, 1 hr, 1.5 hr, 2 hr, 2.5 hr, 3 hr, 3.5 hr, 4 hr, 4.5 hr, 5 hr, 5.5 hr, 6 hr, 6.5 hr, 7 hr, 7.5 hr, 8 hr, 8.5 hr, 9 hr, 9.5 hr, 10 hr, 10.5 hr, 11 hr, 11.5 hr, 12 hr, 12.5 hr, 13 hr, 13.5 hr, 14 hr, 14.5 hr, 15 hr, 15.5 hr, 16 hr, 16.5 hr, 17 hr, 17.5 hr, 18 hr, 18.5 hr, 19 hr, 19.5 hr, 20 hr, 20.5 hr, 21 hr, 21.5 hr, 22 hr, 22.5 hr, 23 hr, 23.5 hr, 24 hr, 24.5 hr, 25 hr, 25.5 hr, 26 hr, 26.5 hr, 27 hr, 27.5 hr, 28 hr, 28.5 hr, 29 hr, 29.5 hr, 30 hr, 30.5 hr, 31 hr, 31.5 hr, 32 hr, 32.5 hr, 33 hr, 33.5 hr, 34 hr, 34.5 hr, 35 hr, 35.5 hr, 36 hr, 36.5 hr, 37 hr, 37.5 hr, 38 hr, 38.5 hr, 39 hr, 39.5 hr, 40 hr, 40.5 hr, 41 hr, 41.5 hr, 42 hr, 42.5 hr, 43 hr, 43.5 hr, 44 hr, 44.5 hr, 45 hr, 45.5 hr, 46 hr, 46.5 hr, 47 hr, 47.5 hr, 48 hr, 48.5 hr, 49 hr, 49.5 hr, 50 hr, 50.5 hr, 51 hr, 51.5 hr, 52 hr, 52.5 hr, 53 hr, 53.5 hr, 54 hr, 54.5 hr, 55 hr, 55.5 hr, 56 hr, 56.5 hr, 57 hr, 57.5 hr, 58 hr, 58.5 hr, 59 hr, 59.5 hr, 60 hr, 60.5 hr, 61 hr, 61.5 hr, 62 hr, 62.5 hr, 63 hr, 63.5 hr, 64 hr, 64.5 hr, 65 hr, 65.5 hr, 66 hr, 66.5 hr, 67 hr, 67.5 hr, 68 hr, 68.5 hr, 69 hr, 69.5 hr, 70 hr, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 2 months, 3 months, 4 months, 5 months, 6 months, 7 months, 8 months, 9 months, 10 months, 11 months, and 1 year, or any ranges therebetween.

In certain embodiments, the molecular iodine-infused polymer or article has at least a 0.45 log, 0.776 log, 1 log, 1.639 log, 2 log, 3 log, 3.06 log, 3.14 log, 3.23 log, 3.39 log, 3.55 log, 4 log, 4.471 log, 5 log, 5.833 log, or 6 log biocidal activity for the time period. In certain embodiments, the molecular iodine-infused polymer or article has at least a 0.45 log, 0.776 log, 1 log, 1.639 log, 2 log, 3 log, 3.06 log, 3.14 log, 3.23 log, 3.39 log, 3.55 log, 4 log, 4.471 log, 5 log, 5.833 log, or 6 log biocidal persistent activity for the time periods.

### V. Other Properties of the Molecular Iodine-Infused Polymer or Article

In certain embodiments, the shelf-lives of the molecular iodine-infused polymers, molecular iodine-infused articles or molecular iodine-infused products are at least about 1 month, 2 months, 3 months, 4 months, 5 months, 6 months, 7 months, 8 months, 9 months, 10 months, 11 months, 1 year, 1.5 years, 2 years, 2.5 years, 3 years, 3.5 years, 4 years, 4.5 years, or 5 years, or ranges therebetween. In certain embodiments, the high molecular iodine-infused polymers, molecular iodine-infused articles or molecular iodine-infused products are stored at room temperature.

### VI. Preparation Methods of Embodiments of Molecular Iodine-Infused Polymer, Article or Product

Embodiments of preparation methods of the molecular iodine-infused polymers or articles are disclosed.

In certain embodiments, the method for preparing the molecular iodine-infused polymer or article comprises iodinating a polymer or article. In certain embodiments, the iodinating step comprises mixing a high molecular iodine concentration composition with the polymer or the article. In certain embodiments, the iodinating step comprises contacting one or more pieces of solid iodine with the polymer or the article. In certain embodiments, the one or more pieces of solid iodine are placed with the polymer or the article. In certain embodiments, the iodinating step comprises contacting an iodine vapor with the polymer or the article.

In certain embodiments, the method for preparing the molecular iodine-infused polymer or article comprises iodinating one or more pre-polymerization compositions of the polymer or the article to provide one or more iodine-pre-polymerization mixtures, and then polymerizing the one or more iodine-pre-polymerization mixtures. In certain embodiments, polymerizing the one or more iodine-pre-polymerization mixtures provides the molecular iodine-infused polymer or article. In certain embodiments, the method for preparing the molecular iodine-infused polymer or article requires additional one or more polymerization steps. In certain embodiments, the pre-polymerization composition may be a composition comprising one or more components selected from the group consisting of monomers, oligomers, crosslinkers, and polymerization initiation compositions (e.g., initiators, catalysts). In certain embodiments, polymerization also includes crosslinking. In certain embodiments, the polymer or article is prepared by mixing two or more pre-polymerization compositions that are the same or different, at least one of the two or more pre-polymerization compositions is iodinated before a polymerization step. Optionally, two or more of the two or more pre-polymerization compositions are iodinated before the polymerization step by the same or different iodination methods.

In certain embodiments, the polymerization step comprises contacting the iodine-pre-polymerization mixture with one or more initiators that initiate and/or one or more catalysts that catalyze the polymerization. In certain embodiments, the polymer is silicone. In certain embodiments, the polymer is silicone and the polymerization step comprising contacting the iodine-pre-polymerization mixture with a platinum catalyst, e.g., as shown in Scheme 1A. In certain embodiments, the polymer is a RTV silicone prepared as shown in Scheme 1B.

In certain embodiments, the method for preparing the molecular iodine-infused polymer or article comprises multiple polymerization steps, the one or more pre-polymerization compositions that is/are iodinated may be one or more composition before any of the multiple polymerization steps. The method for preparing the molecular iodine-infused polymer or article further comprises performing the multiple polymerization steps with one or more iodine-pre-polymerization mixtures and/or non-iodinated pre-polymerization compositions to provide the molecular iodine-infused polymer or article. In certain example, the polymerization step is a crosslinking step.

For example, a molecular iodine-infused high crosslink density silicone can be prepared by iodinating one or more pre-crosslinking compositions of the high crosslink density silicone to provide one or more iodine-pre-polymerization mixtures and then crosslinking the one or more iodine-pre-polymerization mixtures to provide the high crosslink density silicone.

Alternatively, the molecular iodine-infused high crosslink density silicone can be prepared by iodinating one or more pre-crosslinking composition of a first low crosslink density silicone to provide one or more iodine-pre-polymerization mixtures, crosslinking the one or more iodine-pre-polymerization mixtures to provide the molecular iodine-infused low crosslink density silicone, and further crosslinking the molecular iodine-infused low crosslink density silicone to provide the molecular iodine-infused high crosslink density silicone.

In certain embodiments, the iodinating step comprises mixing a high molecular iodine concentration composition with the pre-polymerization composition of the polymer or the article. In certain embodiments, the iodinating step comprises contacting one or more pieces of solid iodine with the pre-polymerization composition of the polymer or the article. In certain embodiments, the iodinating step comprises contacting an iodine vapor with the pre-polymerization composition of the polymer or the article.

In certain embodiments, the method for preparing the molecular iodine-infused polymer or article comprises mixing a high molecular iodine concentration composition with a pre-polymerization composition of the polymer or the article to provide an iodine-pre-polymerization mixture, and then polymerizing the iodine-pre-polymerization mixture to provide the molecular iodine-infused polymer or article.

In certain embodiments, the method for preparing the molecular iodine-infused polymer or article comprises mixing one or more pieces of solid iodine with a pre-polymerization composition of the polymer or the article to provide an iodine-pre-polymerization mixture, and then polymerizing the iodine-pre-polymerization mixture to provide the molecular iodine-infused polymer or article.

In certain embodiments, the method for preparing the molecular iodine-infused polymer or article comprises contacting the polymer or article with an iodine vapor. In certain embodiments, the iodine vapor is provided by one or more pieces of solid iodine that are present in proximity to but without contacting the polymer or article.

In certain embodiments, the method for preparing the molecular iodine-infused polymer or article comprises placing the polymer or article with one or more pieces of solid iodine in a sealed space, wherein the solid iodine is not in contact with the polymer or article, and the sealed space allows iodine vapor generated from the solid iodine to be contained within so that the iodine vapor contacts the polymer or article for iodination.

Embodiments of preparation methods of molecular iodine-infused products are disclosed. In certain embodiments, the method for preparing the molecular iodine-infused product comprises placing the one or more molecular iodine-infused articles in the one or more compartments.

Examples of the molecular iodine concentration of the high molecular iodine concentration composition include, without limitation, about 100 ppm to about 1,120 ppm, about 300 ppm to about 1,274 ppm, about 1,274 ppm to about 170,000 ppm, about 1,274 ppm to about 160,000 ppm, about 1,274 ppm to about 150,000 ppm, about 1,274 ppm to about 100,000 ppm, about 1,274 ppm to about 66,000 ppm, about 1,274 ppm to about 66,000 ppm, about 1,274 ppm to about 15,200 ppm, about 1,400 ppm to about 68,000 ppm, about 1,400 ppm to about 15,200 ppm, about 1,400 ppm to about 66,000 ppm, about 1,500 ppm by weight/volume (wt/v), about 14,000 ppm to about 40,000 ppm, about 15,000 ppm to about 40,000 ppm, about 16,000 ppm to about 38,000 ppm, or about 14,000 ppm to about 20,000 ppm, or ranges therebetween.

Examples of the iodination time (e.g., the contact time of the polymer or article with the high molecular iodine concentration composition, the solid iodine, and/or the iodine vapor) include, without limitation, at least about 30 min., 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 2 days, 3 days, 4 days, 5 days, 6 days, 1 week, eight days, nine days, 10 days, 11 days, 12 days, 2 weeks, 3 weeks, or 4 weeks, or ranges therebetween.

### VII. The High Molecular Iodine Concentration Compositions

In certain embodiments, the high molecular iodine concentration composition comprises:
molecular iodine having concentration of about 387 ppm to about 1,274 ppm, 1,121 ppm to about 1,399 ppm wt/v, about 1,150 ppm to about 1,350 ppm wt/v, about 1,200 ppm to about 1,300 ppm wt/v, or about 1,250 ppm to about 1,275 ppm wt/v, about 1,400 ppm to about 170,000 ppm, about 1,400 ppm to about 160,000 ppm, about 1,400 ppm to about 150,000 ppm, about 1,400 ppm to about 100,000 ppm, about 1,400 ppm to about 68,000 ppm, about 1,400 ppm to about 66,000 ppm, about 1,400 ppm to about 60,000 ppm, about 1,400 ppm to about 55,000 ppm, about 1,400 ppm to about 50,000 ppm, about 1,400 ppm to about 45,000 ppm, about 1,400 ppm to about 40,000 ppm, about 1,400 ppm to about 35,000 ppm, about 1,400 ppm to about 30,000 ppm, about 1,400 ppm to about 25,000 ppm, about 1,400 ppm to about 20,000 ppm, about 1,400 ppm to about 15,200 ppm, about 1,400 ppm to about 15,000 ppm, about 1,400 ppm to about 10,000 ppm, about 1,400 ppm to about 8,703 ppm, about 1,400 ppm to about 8,000 ppm, about 1,400 ppm to about 5,000 ppm, about 1,400 ppm to about 4,077 ppm, about 1,400 ppm to about 2,000 ppm, about 1,400 ppm to about 1,700 ppm, about 1,274 ppm to about 170,000 ppm, about 1,274 ppm to about 160,000 ppm, about 1,274 ppm to about 150,000 ppm, about 1,274 ppm to about 100,000 ppm, about 1,274 ppm to about 68,000 ppm, about 1,274 ppm to about 66,000 ppm, about 1,274 ppm to about 60,000 ppm, about 1,274 ppm to about 55,000 ppm, about 1,274 ppm to about 50,000 ppm, about 1,274 ppm to about 45,000 ppm, about 1,274 ppm to about 40,000 ppm, about 1,274 ppm to about 35,000 ppm, about 1,274 ppm to about 30,000 ppm, about 1,274 ppm to about 25,000 ppm, about 1,274 ppm to about 20,000 ppm, about 1,274 ppm to about 15,200 ppm, about 1,274 ppm to about 15,000 ppm, about 1,274 ppm to about 10,000 ppm, about 1,274 ppm to about 8,703 ppm, about 1,274 ppm to about 8,000 ppm, about 1,274 ppm to about 5,000 ppm, about 1,400 ppm to about 4,077 ppm, about 1,274 ppm to about 2,000 ppm, about 1,274 ppm to about 1,700 ppm, about 1,500 ppm, about 2,000 ppm, about 4,077 ppm, about 4,100 ppm, about 8,200 ppm, about 8,703 ppm, about 15,200 ppm, about 16,500 ppm, about 33,000 ppm, about 14,000 ppm to about 40,000 ppm, about 15,000 ppm to about 40,000 ppm, about 16,000 ppm to about 38,000 ppm, or about 14,000 ppm to about 20,000 ppm by wt/v; and
an organic carrier having a concentration of no less than about 93.5% wt, no less than about 95% wt, no less than about 98% wt, or no less than about 99% wt of the total weight of the high molecular iodine concentration composition.

In certain embodiments, the organic carrier of the high molecular iodine concentration composition is a pharmaceutically acceptable organic carrier, and the high molecular iodine concentration composition is a pharmaceutical formulation. In certain embodiments, the high molecular iodine concentration pharmaceutical formulation further comprises a second pharmaceutically acceptable carrier.

In certain embodiments, the high molecular iodine concentration composition or pharmaceutical formulation is a solution, a viscous solution, a cream, a lotion, a gel, an ointment, a spray, or a suspension. Examples of the high molecular iodine concentration composition or pharmaceutical formulation include the examples disclosed in U.S. Pat. No. 11,596,648, US Patent Application Publication No. 2022/0211751A1, US Patent Application Publication No. 20220211751A1, and US Patent Application Publication No. 20230181801A1, which are incorporated herein by reference in their entireties.

In certain embodiments, the organic carrier has a boiling point at least about 250° C. In certain embodiments, the organic carrier is a weak solvent for the polymer and/or the pre-polymerization composition. In certain embodiments, the organic carrier swells the polymer and/or the pre-polymerization composition when in contact.

In certain embodiments, the organic carrier comprises one or more anhydrous organic solvents. In certain embodiments, the organic carrier is anhydrous.

In certain embodiments, the organic carrier is triacetin.

In certain embodiments of the high molecular iodine concentration compositions or pharmaceutical formulations, at least about 20%, at least about 35%, at least about 55%, at least about 90% of all iodine species is molecular iodine.

In certain embodiments, the high molecular iodine concentration compositions or pharmaceutical formulations is non-aqueous. In certain embodiments, the high molecular iodine concentration compositions or pharmaceutical formulations is substantially non-aqueous. In certain embodiments, the high molecular iodine concentration compositions or pharmaceutical formulations is water-free. In certain embodiments, the high molecular iodine concentration compositions or pharmaceutical formulations is substantially water-free.

In certain embodiments, the high molecular iodine concentration compositions or pharmaceutical formulations has a water content of no more than about 5% wt, no more than about 4.5% wt, no more than about 4% wt, no more than about 3.5% wt, no more than about 3% wt, no more than about 2.5% wt, no more than about 2% wt, no more than about 1.9% wt, no more than about 1.8% wt, no more than about 1.7% wt, no more than about 1.6% wt, no more than about 1.5% wt, no more than about 1.4% wt, no more than about 1.3% wt, no more than about 1.2% wt, no more than about 1.1% wt, no more than about 1% wt, no more than about 0.9% wt, no more than about 0.8% wt, no more than about 0.7% wt, no more than about 0.6% wt, no more than about 0.5% wt, no more than about 0.4% wt, no more than about 0.3% wt, no more than about 0.2% wt, or no more than about 0.1% wt of the total weight of the high molecular iodine concentration compositions or pharmaceutical formulations.

In certain embodiments, the high molecular iodine concentration compositions or pharmaceutical formulations are alcohol-free. In certain embodiments, the high molecular iodine concentration compositions or pharmaceutical formulations are substantially alcohol-free.

In certain embodiments, the high molecular iodine concentration compositions or pharmaceutical formulations has an alcohol content of no more than about 50% wt, no more than about 40% wt, no more than about 30% wt, no more than about 20% wt, no more than 10% wt, no more than about 5% wt, no more than about 4.5% wt, no more than about 4% wt, no more than about 3.5% wt, no more than about 3% wt, no more than about 2.5% wt, no more than about 2% wt, no more than about 1.9% wt, no more than about 1.8% wt, no more than about 1.7% wt, no more than about 1.6% wt, no more than about 1.5% wt, no more than about 1.4% wt, no more than about 1.3% wt, no more than about 1.2% wt, no more than about 1.1% wt, no more than about 1% wt, no more than about 0.9% wt, no more than about 0.8% wt, no more than about 0.7% wt, no more than about 0.6% wt, no more than about 0.5% wt, no more than about 0.4% wt, no more than about 0.3% wt, no more than about 0.2% wt, or no more than about 0.1% wt of the total weight of the high molecular iodine concentration compositions or pharmaceutical formulations.

In certain embodiments, the high molecular iodine concentration composition or pharmaceutical formulation is complexed iodine-free. In certain embodiments, the high molecular iodine concentration composition or pharmaceutical formulation is substantially complexed iodine-free.

In certain embodiments, the high molecular iodine concentration compositions or pharmaceutical formulation has a total concentration of complexed iodine is no more than about 10% of all iodine species, no more than about 25% of all iodine species, no more than about 50% of all iodine species, no more than about 75% of all iodine species, or no more than about 90% of all iodine species.

### VIII. Uses of the Molecular Iodine-Infused Polymers, Molecular Iodine-Infused Articles, or Molecular Iodine-Infused Products.

Embodiments of the molecular iodine-infused polymers, molecular iodine-infused articles, and molecular iodine-infused products have not only the known uses for molecular iodine, but also unexpected uses due to the extended and sustained release and delivery of molecular iodine.

### A. Killing or Inhibiting the Growth of Microorganism

Another aspect of the invention provides a method of killing or inhibiting the growth of a microorganism comprising contacting the microorganism with one or more of the molecular iodine-infused polymers or molecular iodine-infused articles or molecular iodine-infused products disclosed herein.

Another aspect of the invention provides a method of treating or preventing a condition associated with a microorganism in a subject comprising contacting the subject with one or more of the molecular iodine-infused polymers or molecular iodine-infused articles or molecular iodine-infused products.

In certain embodiments, the one or more of the molecular iodine-infused polymers or molecular iodine-infused articles remain in the subject for a time period that the one or more of the molecular iodine-infused polymers or molecular iodine-infused articles have one or more biocidal effects selected from the group consisting of biostatic persistent, biocidal persistent, and prolonged biocidal activities. In certain embodiments, the one or more of the molecular iodine-infused polymers or molecular iodine-infused articles remain in the subject for a time period that the one or more of the molecular iodine-infused polymers or molecular iodine-infused articles have an iodine flux that is sufficient to afford biostatic persistent, biocidal persistent, and prolonged biocidal activity. In certain embodiments, the one or more of the molecular iodine-infused polymers or molecular iodine-infused articles remain in the subject for at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 2 months, or 3 months.

In certain embodiments, the microorganism is killed at the contact site or in vicinity of the contact site. In certain embodiments, the growth rate of the microorganism is reduced at the contact site or in vicinity of the contact site.

In certain embodiments, the method further comprises delivering a therapeutically effective amount or prophylactically effective amount of molecular iodine, high molecular iodine concentration composition, or high molecular iodine concentration pharmaceutical formulation via the molecular iodine infused polymers or articles. In certain embodiments, the method further comprising delivering a molecular iodine in-use composition to the subject via the one or more of the molecular iodine-infused polymers or molecular iodine-infused articles. For example, the molecular iodine in-use composition may be delivered through the molecular iodine-infused catheter. In certain embodiments, the delivery happens after the one or more of the molecular iodine-infused polymers or molecular iodine-infused articles are placed in the subject. In certain embodiments, the delivery happens multiple times at the same interval or at different intervals between each delivery. In certain embodiments, the delivery happens at least 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 5 weeks, 6 weeks, 7 weeks, 8 weeks, 9 weeks, 2 months, or 3 months after the one or more of the molecular iodine-infused polymers or molecular iodine-infused articles are placed in the subject.

In certain embodiments, the microorganism is present at the contact site or in vicinity of the contact site in or on the subject. In certain embodiments, a biofilm is present at the contact site or vicinity of the contact site. In certain embodiments, more than one species of microorganism are present at the contact site or vicinity of the contact site.

Examples of the conditions treatable or preventable include, without limitation, infections caused by microorganism, e.g., urinary tract infection, breast implant infections, wound infections. In certain embodiments, the infection is a chronic infection. In certain embodiments, the infection is resistant infection. In certain embodiments, the infection is a chronic urinary tract infection.

In certain embodiments, the subject is a human.

In certain embodiments, the condition to be treated or prevented is a tissue condition associated with the microorganism. In certain embodiments, the tissue is a mucosal tissue or a cutaneous tissue.

In certain embodiments, the mucosal tissue surrounds or is in a biological cavity. Examples of biological cavities include eye cavity, ear cavity, oral cavity, nasal cavity, vaginal cavity, rectal cavity, and urethral cavity.

In certain embodiments, the microorganisms to be killed of growth of which to be inhibited include one or more species selected from the group consisting of Candida albicans, C. freundii, Escherichia coli, E. faecalis, Enterococcus faecium, Klebsiella pnuemoniae, Proteus mirabilis, Pseudomonas aeruginosa, Staphylococcus aureus, or Staphylococcus epidermidis.

In certain embodiments, the microorganisms to be killed of growth of which to be inhibited include one or more species selected from the group consisting of S. aureus, P. aeruginosa, E. coli, E. faecalis, and C. freundii.

In certain embodiments, the microorganisms to be killed of growth of which to be inhibited include Staphylococcus aureus.

Examples of the microorganism to be killed or growth of which to be inhibited include virus, bacteria, fungus, and protozoa.

Examples of bacteria to be killed or growth of which to be inhibited include gram-positive and gram-negative bacteria, e.g., Bacillus oleronius, C. freundii, E. faecalis, Enterococcus faecium, Klebsiella pnuemoniae. Proteus mirabilis, Pseudomonas aeruginosa, Staphylococcus aureus, Streptococcus pyogenes, Erysipelothrix rhusiopathiae, Mycobacterium tuberculosis, Mycobacterium bovis, Escherichia coli, Extended Spectrum Beta Lactamase resistant E. coli (ESBL), Shigella flexneri, Staphylococcus aureus, Staphylococcus epidermidis, Serratia marcescens, Vibrio cholera, MRSA, Salmonella enterica, Gonorrhea, Syphilis, Shewanella algae, Shewanella putrefaciens, Chlamydia, Chlamydia trachomatis, Chlamydia pneumoniae, Chlamydia psittacci, Aeromonas hydrophila, Vibrio species, Pasteurella multocida, Stapylococcus species, Corynebacterium species, Pripionibacterium species, and antibiotic resistant bacteria, e.g., antibiotic resistant flesh eating bacteria.

Examples of conditions associated with bacteria to be treated or prevented include tuberculosis, periodontitis, acne (e.g., Propionibacterium acnes), rosacea, impetigo, cellulitis, folliculitis, blepharitis (e.g., anterior blepharitis, posterior blepharitis, rosacea blepharitis), bacterial conjunctivitis, blepharoconjunctivits, bacterial corneal ulceration, post-operative endophthalmitis, endophthalmitis after intravitreal or intracameral injection, and infections caused by the bacterium (e.g., urinary tract infections).

Examples of fungus to be killed or growth of which to be inhibited include Apophysomyces variabilis, Aspergillus, Basidiobolus ranarum, Blastomyces dermatitidi, Coccidioides (e.g., Coccidioides posadasii, Coccidioides immitis), Conidiobolus (e.g., Conidiobolus coronatus, Conidiobolus incongruous), Epidermophyton, Fonsecaea (e.g., Fonsecaea pedrosoi, Fonsecaea compacta), Fusarium, Geotrichum candidum, Herpotrichiellaceae (e.g., Exophiala jeanselmei), Histoplasma (e.g., Histoplasma capsulatum, Histoplasma duboisii), Hortaea werneckii, lacazia (e.g., Lacazia loboi), Hyalohyphomycosis, Lichtheimia corymbifera, Malassezia furfur, Microsporum (e.g., Microsporum canis, Microsporum gypseum), Mucor indicus, onychomycosis (e.g., Distal subungual onychomycosis, Proximal subungual onychomycosis), Phialophora verrucose, Piedraia hortae, Pityrosporum, Pseudallescheria boydii, Rhizopus oryzae, Sporothrix schenckii, Syncephalastrum racemosum, Talaromyces marneffei, Trichophyton (e.g., Trichophyton mbmm, Trichophyton mentagrophytes), and yeast (e.g., Candida such as Candida albicans, Candida glabrata, Candida tropicalis, Candida lusitaniae; Cryptococcus neoformans; Pneumocystis such as Pneumocystis jirovecii).

Examples of conditions associated with fungus to be treated or prevented are Alternariosis, black Piedra, blastomycosis, chromoblastomycosis, conidiobolomycosis, favus, fungal folliculitis, fungal corneal ulceration, Lobomycosis, onychomycosis, Otomycosis, Phaeohyphomycosis Pityrosporum folliculitis, ringworm, tinea (e.g., tinea pedis, tinea cruris, tinea barbae, tinea manuum, tinea unguium, tinea unguium, tinea faciei, tinea versicolon, tinea nigra, tinea corporis gladiatorum, tinea imbricate, tinea incognito), yeast infection (e.g., seborrheic dermatitis, vaginal yeast infections).

Examples of protozoa to be killed or growth of which to be inhibited include Acanthamoeba, Leishmania parasites, trypanosoma, Entamoeba histolytica, and Toxoplasma gondii.

Examples of conditions associated with protozoa to be treated or prevented include Acanthamoeba infections (e.g., Acanthamoeba corneal ulceration), Acanthamoeba keratitis, Leishmaniasis, trypanosomiases, Amebiasis, and Toxoplasmosis.

### B. Uses on Wound-Healing or Scar-Prevention

Another aspect of the invention relates to a method of fostering wound-healing or preventing a scar of a subject comprising contacting the wound with one or more of the molecular iodine-infused polymers or molecular iodine-infused articles (e.g., wound dressing, sutures, artificial skin) or molecular iodine-infused products.

In certain embodiments, the molecular iodine-infused polymer or article is applied to a wound or tissue in proximity of the wound. In certain embodiments, the tissue is a mucosal tissue or a cutaneous tissue.

In certain embodiments, the wound to be healed is healed with a scar less severe than a similar wound healed without the treatment. In certain embodiments, the scar is less severe as characterized by one or more improvements, e.g., without limitation, reduction of the scar height, reduction of the scar surface, reduction of the thickness of the scar, improvement of the pliability of the scar, improvement of the texture of the scar, reduction of pigmentation of the scar, and reduction of vascularity of the scar. See, e.g., Fearmonti et al., “A Review of Scar Scales and Scar Measuring Devices,” Eplasty, 2010:10 e43, which is incorporated by reference (www.ncbi.nlm.nih.gov/pmc/articles/PMC2890387/). In certain embodiments, the one or more improvements of the scar are at least about 10%, about 10% to about 100%, at least about 20%, at least about 30%, at least about 40%, at least about 50%, at least about 60%, at least about 70%, at least about 80%, at least about 90%, at least about 95%, or at least about 99%. In certain embodiments, the reduction of the scar height, the reduction of the scar surface, or the reduction of the thickness of the scar are/is at least about 10%, about 10% to about 100%, at least about 20%, at least about 30%, at least about 40%, at least about 50%, at least about 60%, at least about 70%, at least about 80%, at least about 90%, at least about 95%, or at least about 99%.

In certain embodiments, the wound to be healed in presence of the molecular iodine-infused polymer or article is healed without a visible scar.

In certain embodiments, the wound to be healed in presence of the molecular iodine-infused polymer or article is healed in a shorter time period in the presence of the molecular iodine-infused polymer or article compared to similar wound healed without the presence of the molecular iodine-infused polymer or article.

In certain embodiments, the wound to be healed in presence of the molecular iodine-infused polymer or article is inflicted by a cut, a friction, cold, heat, radiation (e.g., sunburn), a chemical, electricity, a microorganism infection, pressure, or a condition of the subject (e.g., diabetes). For example, in certain embodiments, the subject is diabetic (i.e., a subject having a condition, and the condition is diabetes). Examples of wounds in a diabetic subject include bullosis diabeticorum (diabetic blisters), eruptive xanthomatosis, and neuropathic ulcers (e.g., diabetic foot ulcers). In certain embodiments, the wound is a decubitus ulcer (i.e., pressure ulcer, pressure sore, or bedsore). In certain embodiments, the wound is a burn wound.

### C Uses for Pre-, Post- and Intra-Operative Infection Prevention

Provided are embodiments of a method of treating a surgical site to foster healing, or prevent infection in a subject in need comprising contacting the subject with one or more of the molecular iodine-infused polymers or molecular iodine-infused articles (e.g., wound dressing, sutures, and artificial skin) or molecular iodine-infused products. In certain embodiments, the method of treating a surgical site is performed as a pre-surgical treatment, a post-surgical treatment, or a treatment during a surgery.

A surgical site refers to an incision site on a subject, or any part of a subject's anatomy in organs or spaces which were opened or manipulated during a surgery. In certain embodiments, the incision site includes a superficial incision site, e.g., involving skin or subcutaneous tissue. In certain embodiments, the incision site includes a deep incision site, e.g., in tissues deeper than the skin or subcutaneous tissue, such as fascial and muscle layers.

In certain embodiments, the molecular iodine-infused polymer or article is applied to a surgical site or tissue in proximity of the surgical site.

In certain embodiments, the molecular iodine-infused polymer or article is applied to a surgical site pre-surgical, post-surgical, or during surgery. Examples of surgery include pleurodesis procedure, cytoreductive surgery, thoracic surgery, esophageal resection, complete resection or pleural reductive surgery for thymoma, primary functional endoscopic sinus surgery, spinal surgery, and colonic resection.

In certain embodiments, the molecular iodine-infused polymer or article is administered as an eyelid antisepsis prior, during, or after an eye surgery (e.g., cataract surgery) or other procedures on or proximate to an eye (e.g., intravitreal injection, intracameral injection). In certain embodiments, the eyelid antisepsis is applied to eyelid by hand or by the molecular iodine-infused polymer or molecular iodine-infused article (e.g., an eyelid wipe).

In certain embodiments, the molecular iodine-infused polymer or article is applied to a surgical site comprising a chest cavity (i.e., a space between a subject's lung and chest wall). For example, the molecular iodine-infused polymer or molecular iodine-infused article may be administered before, during, or after a pleurodesis procedure in a subject in need.

### D. Uses for Sanitizing One or More Articles

Provided herein are embodiments of a method of sanitizing one or more articles or materials (e.g., metals, polymers, fabrics, plants, and food) comprising placing the one or more articles or materials adjacent to one or more molecular iodine-infused polymers or one or more molecular iodine-infused articles. In certain embodiments, the one or more articles or materials are placed in a sealed compartment comprising the one or more molecular iodine-infused polymers or one or more molecular iodine-infused articles. In certain embodiments, the sealed compartment is made of the one or more molecular iodine-infused polymers. In certain embodiments, the sealed compartment comprises one or more molecular iodine-infused polymers or articles placed therein.

### IX. Preparation of High Molecular Iodine Concentration Compositions

Provided are also methods for preparing the high molecular iodine concentration compositions. In certain embodiments, the high molecular iodine concentration composition is prepared by dispersing molecular iodine into the organic carrier.

In certain embodiments, all organic solvents are anhydrous.

Examples of the organic carrier include, without limitation, triacetin.

The above disclosure and the following examples are illustrative of the teachings of this application and are not meant to limit the scope and application of the invention. The examples and embodiments are for illustrative and will suggest modifications to persons skilled in the art which are included within the spirit and purview of this application and scope of the appended claims.

### EXAMPLES

### Example 1. Preparation of Embodiments of the High Molecular Iodine Concentration Composition

A triacetin-iodine solution was prepared by dissolving molecular iodine in triacetin and stirring it at room temperature. After ten days of constant gentle mechanical mixing, one part iodine was mixed with four parts triacetin, and the composition had a molecular iodine concentration of approximately 38,000 ppm. Additional embodiments of the high molecular iodine concentration composition with different molecular iodine concentrations were prepared by diluting the composition having a molecular iodine concentration of approximately 38,000 ppm.

### Example 2. Preparation of Embodiments of the Molecular Iodine-Infused Polymer Using Embodiments of High Molecular Iodine Concentration Composition (e.g., Triacetin-Iodine Solutions Prepared According to Example 1, and/or High Molecular Iodine Concentration Compositions Disclosed, e.g., in WO2018017645A1, WO2022036280A1, WO2023019033A1, And WO2023108160A1, which are Incorporated Herein by Reference in their Entireties)

Any platinum cure two-part (Part A and Part B) silicone rubber can be utilized. As an example, Solaris™ silicone (by Smooth-On) was tested which was a low viscosity, clear and colorless liquid platinum cure silicone rubber compound designed for use in protecting of electronic components and other assemblies. The silicone may protect physical components against shock, vibration, moisture, ozone, dust, chemicals and other environmental hazards.

Before casting, pre-mix Part B thoroughly. A triacetin-iodine solution prepared according to Example 1 could be added to Part A and/or Part B during mixing or after mixing both parts. The triacetin-iodine solution may have a molecular iodine concentration of about 14,000 ppm to about 20,000 ppm, or at least about 15,000 ppm. The silicone rubber may be cast in any laboratory Petri dishes or other molds of specific size and shape with preferred materials of construction like polystyrene, Teflon, polypropylene, polyethylene or glass.

In one example, in a 100 mm by 15 mm Petri dish, 6 grams of Parts A and B were added and mixed thoroughly for 3 minutes. After mixing thoroughly for 3 minutes, the mixture was transferred into a second clean Petri dish and mixed again for another 2 to 3 minutes before pouring into a third same size petri dish. Vacuum degassing was conducted to eliminate entrapped air. The mixture was subjected to 29 inches of mercury in a vacuum chamber until the mixture rose, broke and fell. The same process was repeated 3 to 4 times. Then the rubber was allowed to cure for 24 hours at room temperature (73° F./23° C.) before demolding. It was not recommended to cure the rubber where temperature is less than 65° F./18° C.

After the curing process was done, the silicone rubber was removed from the Petri dish by breaking the sides of the plastic mold and peeling the rubber from the bottom of the mold free. A transparent circular monolith rubber formed.

Molecular iodine could be diffused into the monolithic rubber into the triacetin-iodine solution.

In a suitable sealed container, the cured silicone monolith could be immersed in triacetin-iodine solution (e.g., as prepared according to the methods disclosed in Example 1) for at least 24 hours at temperatures ranging from 15 to 20 degrees Celsius. The immersion time could be extended depending on the size and thickness of the rubber, but the first 24 hours seemed to be sufficient to allow molecular iodine to diffuse at least a micron deep into the surface. The rubber was removed, and any excess liquid on the surface was removed with a soft tissue. If the rubber is not sealed and is left in ambient conditions, a longer period of time (e.g., greater than 48 hours) may be needed, depending on the crosslink density, immersion time, and swelling conditions.

The weight concertation of triacetin with respect to the total silicone does not exceed 0.2% of the total silicone system weight in order to partially or slightly inhibit crosslinking so that the rubber swells easier for more iodine uptake during the immersion process.

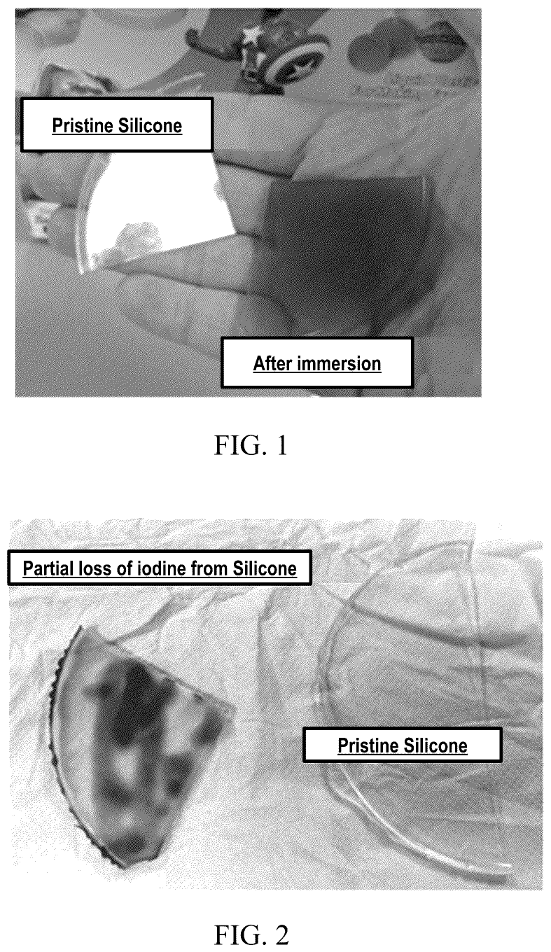

The presence of molecular iodine in the polymerized silicone was observed in the image shown in FIG. 1. The silicone in the upper left side did not contain any iodine and the silicone in the lower right side of the image did and showed a brown color (FIG. 1).

Molecular iodine incorporated into the molecular iodine-infused silicone prepared herein diffused in the silicone polymer and transited out of the polymer. See, e.g., FIG. 2, left silicone showed partial loss of molecular iodine after 24 hours, and the right silicone did not contain molecular iodine.

### Example 3. Preparation of Embodiments of the Molecular Iodine-Infused Silicone Using Room Temperature Vulcanizing Silicones (RTV Silicones) and Embodiments of High Molecular Iodine Concentration Composition (e.g., I2/Glycerin Solutions in Various Concentrations)

Unless specified otherwise, silicone samples used in this example were prepared from commercial hydroxyterminated silicone oil, MeSi(OMe)3 and Bu2Sn(OCOC11H23)2 as catalyst. Silicone samples were prepared as either ‘coupons’ or films. Coupons were cured in 34.6 mm diameter Petri dishes and were ˜2 mm thick (some were of lower diameter and thicker—see below). Films—200 micron thick—were drawn on a Teflon substate using a doctor blade and then cured; each film used in experiments was 4 cm×4 cm×200 microns.

### A. Molecular Iodine Infused Silicone Prepared Using I₂/Glycerin Solutions Pre-Cure

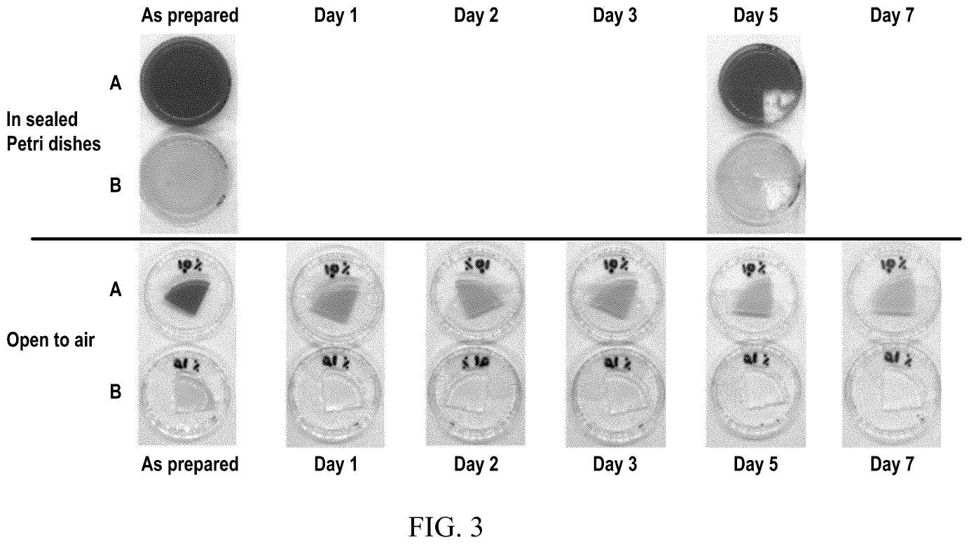

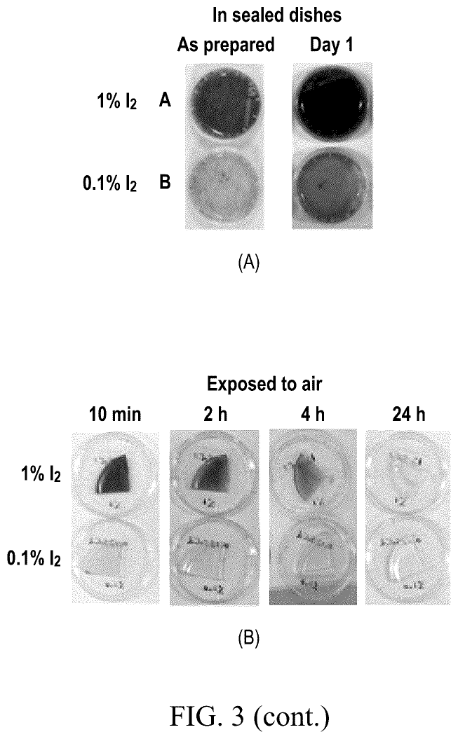

Embodiments of molecular iodine infused silicone samples were prepared by incorporating an I₂/glycerin solution (10 wt % I₂) pre-cure into the silicone samples, and then curing the silicone samples incorporated with I₂/glycerin solution. The I₂/glycerin solution was loaded into uncured silicone at a level of 10 wt % to provide Sample 3(A)-A having ˜1 wt % I₂ (FIG. 3, sample A) and 1 wt % to provide Sample 3(A)-B having ˜0.1 wt % I₂ (FIG. 3, sample B). When the molecular iodine infused silicone samples were sealed to prevent air exchange, no loss of color (an indication of loss of I2) was noted (e.g., FIG. 3, top panel, comparing samples 3(A)-A and 3(A)-B as prepared v. at Day 5). However, I2 release was observed when the samples were exposed to air (e.g., FIG. 3, bottom panel, comparing samples 3(A)-A and 3(A)-B as prepared v. at Day 1, 2, 3, 5 and 7 after opened to air). The I₂ loss from Sample 3(A)-A (the 1 wt % I₂/silicone) over 1 week (FIG. 3, bottom panel), by color, showed that most of the I2 had left, leaving about 0.1% I2, one tenth of the original loading.

The I₂ releases from Samples 3(A)-A and 3(A)-B in the air were much slower than when glycerin was absent from the system, suggesting glycerin and related molecules (e.g., without limitation, polyol, e.g., without limitation, propylene glycol, and compounds with multiple ester groups, e.g., without limitation, triacetin) could be used to tune slow release of I2 from RTV silicone elastomers. For example, diffusing I₂ into silicone rubber with such carriers may enhance or speed up I₂ release from the molecular iodine-infused silicone rubbers. Without wishing to be bound by any theory, such enhancement of I₂ release may be via physical leaching of the liquid or solvent from the silicone rubber matrix that carries I₂ along.

B. Molecular Iodine Infused Silicone Prepared Using Solid I₂ During Cure, Low Doses: 1 wt % and 0.1 wt % I₂.

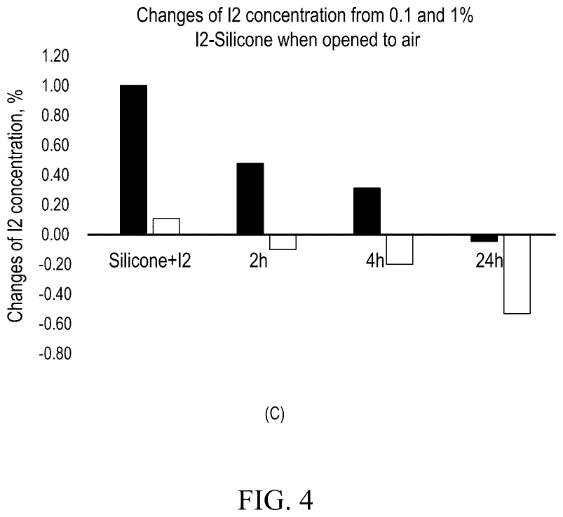

Embodiments of molecular iodine infused silicone samples were prepared by incorporating powdered I₂ to uncured silicone and then curing the silicone loaded with I₂. Solid I₂ did not substantially change the physical properties of the cured molecular iodine infused silicone samples. The molecular iodine infused silicone samples prepared using solid I₂ were quite similar to the molecular iodine infused silicone samples prepared using I₂/glycerin (e.g., as described in Example 3(A) above), except with a much faster spontaneous release than the molecular iodine infused silicone samples prepared using I₂/glycerin (e.g., as described in Example 3(A)). Migration of I2 throughout the silicone samples was very efficient. Small particles at t=0 mostly disappeared in one day (FIG. 4A, A: sample 3(B)-A (10 wt % I₂), B: sample 3(B)-B (1 wt % I₂)). Most I2 was lost when the samples were exposed to air after about 24 hours (FIGS. 4B and 4C).

### C. Molecular Iodine Infused Silicone Prepared Using Solid I₂ During Cure, High Doses: 50 wt %

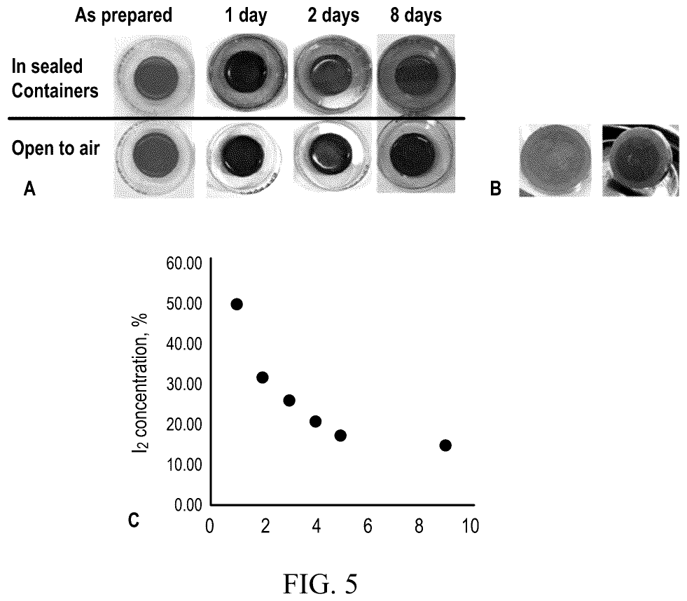

Embodiments of molecular iodine infused silicone samples with high dose (Sample 3(C), up to 50 wt % I₂) were prepared by incorporating powdered I₂ to uncured silicone and then curing the silicone loaded with I₂. These materials also released I₂ rapidly into air (FIGS. 5A and 5C), preferentially from the edges (FIG. 5B). After a week, a residual coloration was found in the silicone. The deep coloration was retained after a much longer time. It continued to fade up to ten days (measurements continued, FIG. 5A).

D. Molecular Iodine Infused Silicone Prepared Using a Single Piece of Solid I₂ after Cure.

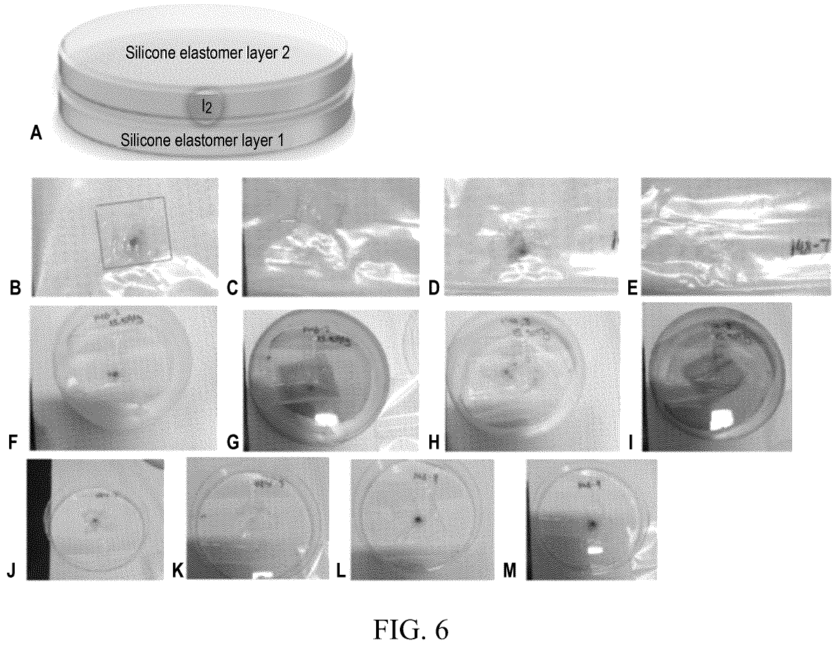

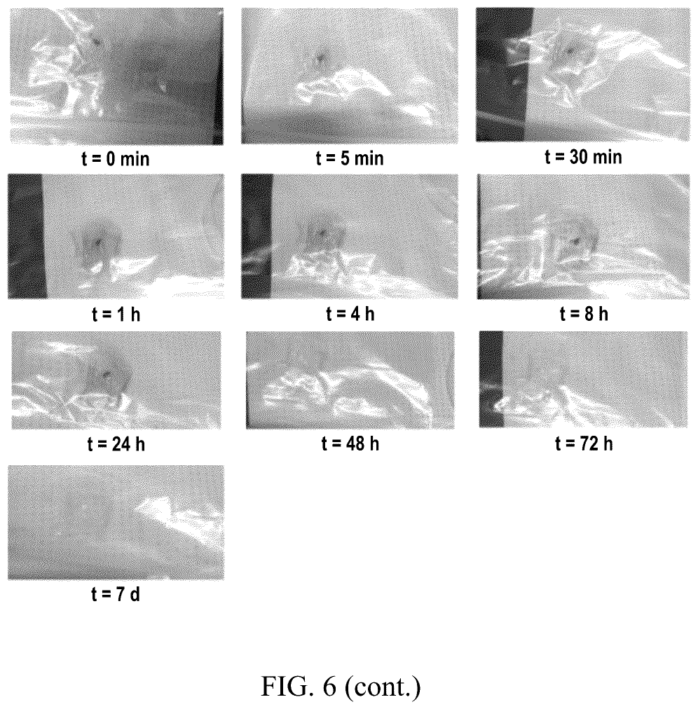

Embodiments of molecular iodine infused silicone samples were prepared by sandwiching a single piece of solid I₂ with two silicone pieces (FIG. 6A) that were low crosslink density (Samples 3(D)-A) or high crosslink density (Samples 3(D)-B). Note that various samples 3(D)-A could have various iodine concentrations, and various samples 3(D)-B could have various iodine concentrations either.

Three different protocols were used to observe the I₂ migrations in the samples: 1) low void space—samples were place in a ziplock bag with very little excess air (FIGS. 6B-6E, Sample 3(D)-A after 5 min and 48 h, and Samples 3(D)-B after 5 min and 48, respectively); 2) samples were placed in a 9 cm diameter Petri dish and sealed at the seam (FIGS. 6F-6I, Sample 3(D)-A after 5 min and 48 h, and Samples 3(D)-B after 5 min and 48, respectively); and 3) samples were left in a Petri dish in open air (FIGS. 6K-6M, Sample 3(D)-A after 5 min and 48 h, and Samples 3(D)-B after 5 min and 48, respectively). At selected time points, the samples were removed, weighed and then resealed in the ziplock bag or Petri dish.

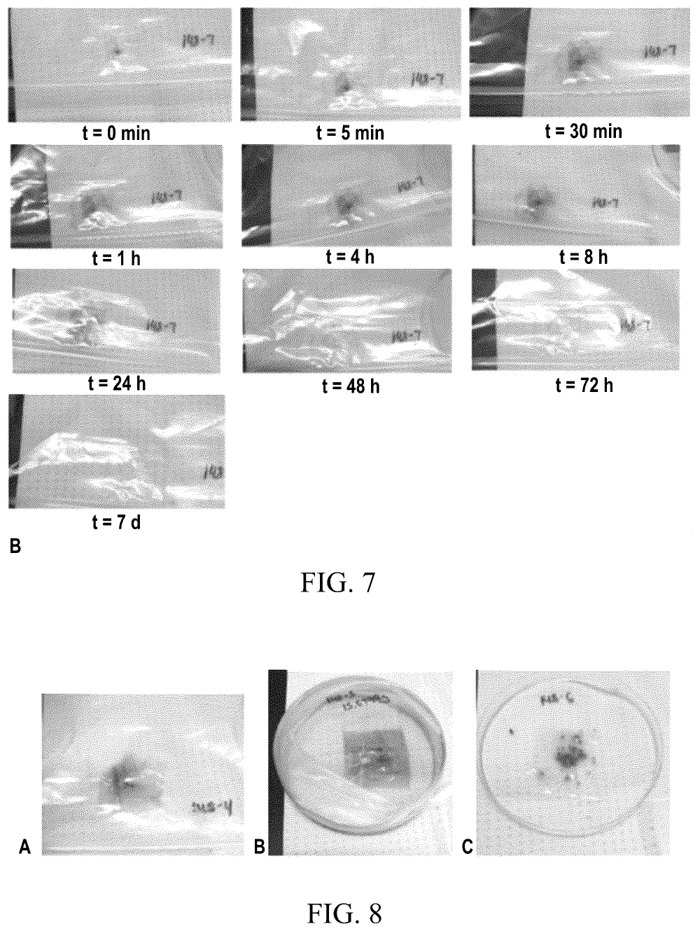

There was little impact of crosslink density on I2 migration (FIGS. 6B&6C vs 6D&6E, see also FIG. 7A v. FIG. 7B). This is advantageous, as it suggests controlled delivery can be targeted independently of the physical properties required for a give applicant (e.g., Young's modulus). When the sample was provided a larger gas area in the surroundings, the iodine rapidly moved laterally through the sample and then partitioned into the gas phase (FIGS. 6F-6I). Similarly, crosslink density was not an important factor for the I₂ migration (FIGS. 6F&6G vs 6H&6I)). When placed in an open Petri dish, diffusion through the shortest path (the thin ‘z’ direction) was followed by migration out of the device at a rate that exceeded lateral (‘xy’) migration (FIGS. 6J-6M).

Additional embodiments of the molecular iodine-infused silicone, samples 3(D)-A (low crosslink density silicone) and 3(D)-B (high crosslink density silicone) were prepared using a single I₂ crystals placed between two layers of precured silicones and observed after placed in a Ziploc bag for 5 min. 30 min, 1 h, 4 h, 8 h, 24 h, 48 h, 72 h, and 7 days (FIG. 7A for Sample 3(D)-A, with 9.7% mass increase in the silicone, and at equilibrium, the iodine mass in the two silicone pieces accounting for 8.8% of the initial solid placed between the two silicone pieces; and FIG. 7B for Sample 3(D)-B, with 10.0% mass increase in the silicone, and at equilibrium, the iodine mass in the two silicone pieces accounting for 11.2% of the initial solid placed between the two silicone pieces).

E. Molecular Iodine Infused Silicone Prepared Using Multiple Piece of Solid I₂ after Cure.

Embodiments of molecular iodine infused silicone samples were prepared by sandwiching multiple piece of solid I₂ with two silicone pieces that were low crosslink density (Samples 3(E)-A) or high crosslink density (Samples 3(E)-B). Note that various samples 3(E)-A could have various iodine concentrations, and various samples 3(E)-B could have various iodine concentrations either.

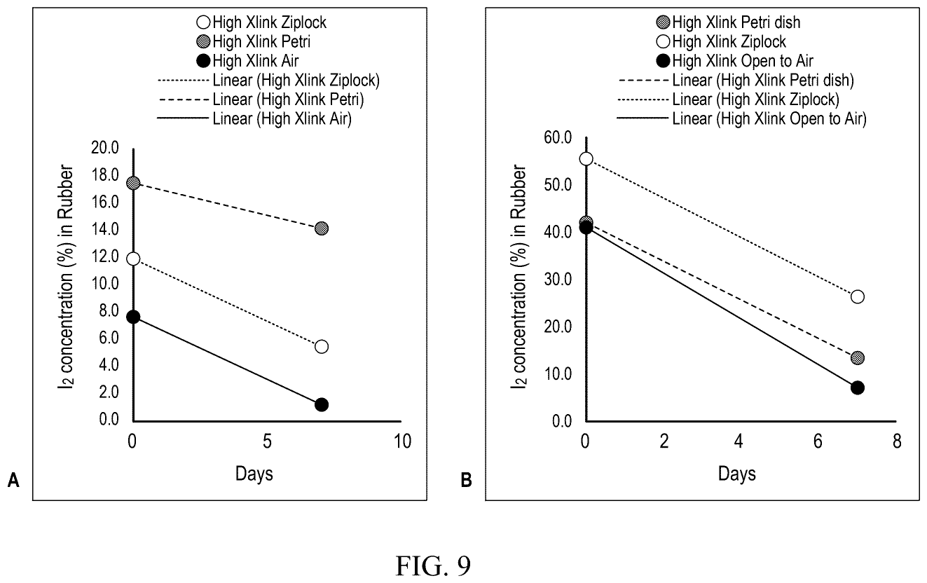

Similar trend of iodine migration was observed for samples prepared using multiple piece of solid I₂, which provided a higher dose of molecular iodine (See, e.g., FIG. 9B, for samples 3(E)-B having I₂ concentration of up to about 40 w % and up to about 60 wt %). Similar efficiency of lateral flow was observed, e.g., for samples 3(E)-A that were placed in a Ziploc (FIG. 8A, after 4 hours) or Petri dish (FIG. 8B, after 4 hours), but not in an open system (FIG. 8C, after 4 hours). These data suggest that an entire silicone device may be iodinated from contacting a solid iodine at a single location within the device and it may take only a few hours for equilibration to occur within the device to prepare the device for use.

I₂ concentrations of high crosslink silicone samples prepared using a single piece of solid iodine (Sample 3(D)-B) placed in Ziplock (FIG. 9A, top circle, High Xlink Ziplock), sealed petri dish (FIG. 9A, middle circle, High Xlink Petri), and open air (FIG. 9A, bottom circle, High Xlink Air) were measured at time 0 and one week.

I₂ concentrations of high crosslink silicone samples prepared using multiple pieces of solid iodine (Sample 3(E)-B) placed in Ziplock (FIG. 9B, middle circle, High Xlink Ziploc), sealed petri dish (FIG. 9B, top circle, High Xlink Petri dish), and open air (FIG. 9B, bottom circle, High Xlink Open to air) were measured at time 0 and one week.

### F. Molecular Iodine Infused Silicone Prepared Using I₂ Vapor and Thick Films.

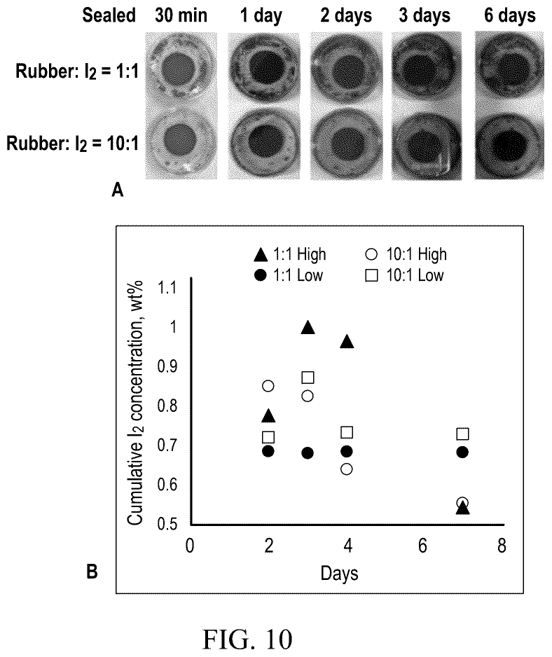

Embodiments of molecular iodine infused silicone samples were prepared by placing solid iodine (about 1:1 or 1/10 mass of iodine to silicone) near, but not in contact with a silicone think firm (2.145 cm diameter, 3.12 mm thick, which could be high crosslink and low crosslink silicones) in a sealed petri dish (3.5 cm diameter). I2 was allowed to equilibrate with the air in the petri dish and with the silicone. About 1 wt % I2 entered the silicone film (FIG. 10B). The samples essentially reached equilibrium I2 concentration after 1 day. The losses of I₂ mass after day 3 may be attributed to continuing curing of the silicon films (which slowly released alcohol; these silicone films were only cured one day before the iodination experiments started). These materials were very dark in color (FIG. 10A).

Exemplary images of samples with lower I₂ ratio treatment (Rubber:I₂=10:1) and higher I₂ ratio treatment (Rubber:I₂=1:1) placed in a sealed Petri dish after 30 min, day 1, 2, 3, or 6 are shown in FIG. 10A. FIG. 10B shows I₂ increases in low crosslink density silicone with higher I₂ ratio treatment (1:1 Low, dark circle), low crosslink density silicone with lower I₂ ratio treatment (10:1 Low, diamond), high crosslink density silicone with higher I₂ ratio treatment (1:1 High, triangle), and high crosslink density silicone with lower I₂ ratio treatment (10:1 high, light circle), and 3(F)-B (Rubber:I₂=10:1) after days of exposure to I₂ vapor.

I₂ concentration of the samples of FIG. 10(A) were observed when the samples were exposed to open air over a few hours. Images of samples with higher I₂ ratio treatment (Rubber:I₂=1:1) and lower I₂ ratio treatment (Rubber:I₂=10:1) after opened to air for 2 h, 4 h, or 24 h showed the dark color faded away significantly in 24 h (FIG. 12A). Bar chart showing the loss of I₂ from samples with higher I₂ ratio treatment (Rubber:I₂=1:1, left bar) and lower I₂ ratio treatment (Rubber:I₂=10:1, right bar) after opened to air for 2 h, 4 h, or 24 h is provided in FIG. 12B.

### G. Molecular Iodine Infused Silicone Prepared Using I₂ Vapor and Thin Films.

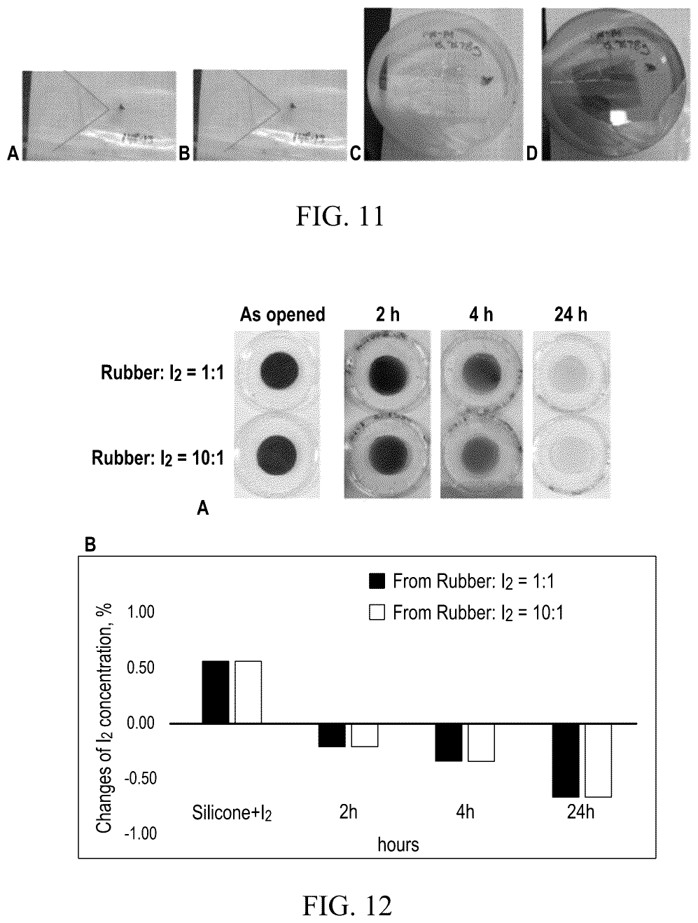

Embodiments of molecular iodine infused silicone samples were prepared by placing solid iodine (about 1:1 or 1/10 mass of iodine to silicone) near, but not in contact with a thin film in a Ziploc bag with very little air volume after 5 min (FIG. 11A) and 48 hrs (FIG. 11B) or in a sealed Petri dish after 5 min (FIG. 11C) and 48 hrs (FIG. 11D).

1. A molecular iodine-infused polymer or article comprising molecular iodine and a polymer or article.

2. The molecular iodine-infused polymer or article of claim 1 having a molecular iodine concentration of at least about 50 wt % or about 30 wt %.

3. The molecular iodine-infused polymer or article of claim 1, having a molecular iodin concentration of at least about 5 wt %, or about 10 wt % after exposition to open air for about 1 day, about 2 days, about 3 days, about 4 days, about 5 days, about 6 days, about 7 days, about 8 days, about 9 days, or about 10 days.

4. The molecular iodine-infused polymer or article of claim 1, having one or more biocidal effects selected from the group consisting of biostatic persistent, biocidal persistent, and prolonged biocidal activity for at least about 1 hour.

5. The molecular iodine-infused polymer or article of claim 1, wherein the molecular iodine-infused polymer is prepared by iodinating a polymer.

6. The molecular iodine-infused polymer or article of claim 1, wherein the molecular iodine-infused polymer or article is prepared by iodinating one or more pre-polymerization compositions of the polymer or article to provide one or more iodine-pre-polymerization mixtures, and then polymerizing the one or more iodine-pre-polymerization mixtures.

7. The molecular iodine-infused polymer or article of claim 5, wherein iodinating the polymer comprises one or more steps selected from the following:
mixing a high molecular iodine concentration composition with the polymer or article, contacting one or more pieces of solid iodine with the polymer or article, and contacting an iodine vapor with the polymer or article.

8. The molecular iodine-infused polymer or article of claim 6, wherein iodinating the one or more pre-polymerization compositions of the polymer or article comprises one or more steps selected from the following:
mixing a high molecular iodine concentration composition with one or more pre-polymerization compositions of the polymer or article, contacting one or more pieces of solid iodine with the one or more pre-polymerization compositions of the polymer or article, and contacting an iodine vapor with the one or more pre-polymerization compositions of the polymer or article.

9. The molecular iodine-infused polymer or article of claim 1, wherein the polymer is silicone.

10. The molecular iodine-infused polymer or article of claim 1, wherein the polymer is RTV silicone.

11. A molecular iodine-infused product comprising one or more molecular iodine-infused polymer or article of claim 1.

12. A method of killing or inhibiting the growth of a microorganism comprises contacting the microorganism the microorganism with the molecular iodine-infused polymer or article of claim 1.

13. A method of treating or preventing a condition associated with a microorganism in a subject comprising contacting the subject with the molecular iodine-infused polymer or article of claim 1.

14. A method of fostering wound-healing or preventing a scar of a subject comprising contacting the wound with the molecular iodine-infused polymer or article (e.g., wound dressing, sutures, artificial skin) of claim 1.

15. A method of treating a surgical site to foster healing, or prevent infection in a subject in need comprising contacting the subject with the molecular iodine-infused polymer or article of claim 1.

16. A method of sanitizing one or more articles or materials comprising placing the one or more articles or materials adjacent to the molecular iodine-infused polymer or article of claim 1.
